(19) United States
(12) Patent Application Publication
(10) Pub. No.: US 2021/0113093 A1
(43) Pub. Date: Apr. 22, 2021

(54) BLOOD PRESSURE ESTIMATION SYSTEM, BLOOD PRESSURE ESTIMATION METHOD, LEARNING METHOD, AND PROGRAM

(71) Applicant: TOSHO ESTATE CO., LTD., Tokyo (JP)

(72) Inventors: Akio Nozawa, Sagamihara-shi (JP); Kosuke Oiwa, Sagamihara-shi (JP); Kenichi Nakano, Tokyo (JP)

(73) Assignee: TOSHO ESTATE CO., LTD., Tokyo (JP)

(21) Appl. No.: 17/011,318

(22) Filed: Sep. 3, 2020

(30) Foreign Application Priority Data
Sep. 3, 2019 (JP) 2019-160646
Jul. 30, 2020 (JP) 2020-129600

(51) Int. Cl. A61B 5/021; A61B 5/00; A61B 5/01; G06K 9/00

(52) U.S. Cl. CPC A61B 5/021; A61B 5/004; A61B 5/7275; G06K 9/00281; A61B 5/015; A61B 5/7239; A61B 5/7267

(57) ABSTRACT

To provide a blood pressure estimation system capable of instantaneously estimating a blood pressure of a subject in a non-contact manner. A blood pressure estimation system includes a face image acquisition unit that acquires a face image of a subject, and a blood pressure estimation unit that estimates a blood pressure of a subject based on a spatial feature amount of the face image.

### TECHNICAL FIELD

The present invention relates to a technique that estimates a blood pressure of a subject in a non-contact manner.

### BACKGROUND ART

A technique that estimates a blood pressure of a subject in a non-contact manner. Patent Literature 1 describes a system that calculates a pulse wave timing from a time change in luminance of a skin image of a subject and calculates a heartbeat timing from a time change in distance between the subject and a receiving antenna to estimate a blood pressure of the subject based on a time difference between the pulse wave timing and the heartbeat timing.

### CONVENTIONAL ART LITERATURE

### Patent Literature

[Patent Literature 1] Japanese Published Unexamined Application No. 2016-77890

### SUMMARY OF THE INVENTION

### Problems to be Solved by the Invention

However, in the technique of Patent Document 1, since, in addition to an imaging means for acquiring a skin image of a subject, a calculating means for calculating a pulse wave timing, a distance measuring means including a receiving antenna for measuring a distance between the subject and the skin, and a calculating means for calculating a heartbeat timing are necessary, a system configuration increases in scale.

In addition, in the technique of Patent Literature 1, since a blood pressure is estimated based on information on a time change in luminance of the skin image of the subject, that is, the temporal feature amount of the skin image, and information on a time change in distance between the subject and the receiving antenna, that is, a temporal feature amount of the position of the skin, a time required to extract the temporal feature amount of the skin image and the temporal feature amount of the skin position is necessary. For this reason, the blood pressure of the subject cannot be instantaneously estimated.

The present invention provides a blood pressure estimation system, a blood pressure estimation method, a learning device, a learning method that can instantaneously estimate a blood pressure of a subject in a non-contact manner, and a program for realizing them using a computer.

### Means for Solving the Problems

In order to solve the problems described above, a blood pressure estimation system is characterized by including a face image acquisition unit that acquires a face image of a subject in a non-contact manner, and a blood pressure estimation unit that estimates a blood pressure of the subject based on a spatial feature amount of the face image.

The blood pressure estimation system acquires the face image of the subject and estimates the blood pressure of the subject based on the spatial feature amount of the face image.

A blood pressure estimation system is characterized in that the blood pressure estimation unit includes a correlation data storage unit that stores correlation data indicating a relationship between a weighted time series of an independent component of the face image and a blood pressure, a spatial feature amount extraction unit that extracts a weighted time series of the independent component of the face image as a spatial feature amount by analyzing the independent component of the face image acquired by the face image acquisition unit, a blood pressure determination unit that determines the blood pressure value corresponding to the weighted time series extracted by the spatial feature amount extraction unit on the basis of the correlation data, and an estimated blood pressure value output unit that outputs the value determined by the blood pressure determination unit as an estimated value of the blood pressure of the subject.

The blood pressure estimation system extracts the weighted time series of the independent components of the face image by analyzing the independent components of the face image of the subject, determines the value of the blood pressure corresponding to the extracted weighted time series from the correlation data, and outputs the value as an estimated value of the blood pressure of the subject.

A blood pressure estimation system is characterized in that the blood pressure estimation unit includes a correlation data storage unit that stores correlation data showing the weighted time series of the independent component of the face image and a relationship between a differential value of the weighted time series and the blood pressure, a weighted time series calculation unit that calculates a weighted time series of the independent components of the face image as the spatial feature amount by analyzing the independent component of the face image acquired by the face image acquisition unit, a weighted time series differential value calculation unit that calculates a differential value of the weighted time series calculated by the weighted time series calculation unit, a blood pressure determination unit that determines a blood pressure value corresponding to the weighted time series calculated by the weighted time series calculation unit and the differential value of the weighted time series calculated by the weighted time series differential value calculation unit from the correlation data, and an estimated blood pressure value output unit that outputs the value determined by the blood pressure determination unit as an estimated value of the blood pressure of the subject.

The blood pressure estimation system calculates the weighted time series of the independent components of the face image by analyzing the independent components of the face image of the subject and calculates a differential value of the weighted time series. The system determines the weighted time series and the blood pressure value corresponding to the differential value from the correlation data, and outputs the value as an estimated value of the blood pressure of the subject.

A blood pressure estimation system is characterized in that the differential value of the weighted time series includes a first-order differential value and a second-order differential value of the weighted time series, and the weighted time series differential value calculation unit calculates the first-order differential value and the second-order differential value of the weighted time series.

The blood pressure estimation system determines, from the correlation data, the weighted time series and the blood pressure values corresponding to the first-order differential value and the second-order differential value of the weighted time series to output the value as an estimated value of the blood pressure of the subject.

A blood pressure estimation system characterized in that the face image is a face thermal image or a face visible image.

The blood pressure estimation system acquires a face thermal image or a face visible image of a subject and estimates the blood pressure of the subject based on a spatial feature amount of the face thermal image or the face visible image.

An infrared thermography is used to acquire the face thermal image. The face thermal image is an image in which infrared rays radiated from the face of the subject are analyzed and the heat distribution is represented as a diagram. A camera popularly used, that is, a device having an optical system for forming an image to capture a video image is used to acquire the face visible image.

A blood pressure estimation system is characterized in which the blood pressure estimation unit includes a determination spatial feature amount storage unit that stores a determination spatial feature amount corresponding to a blood pressure stage consisting of two stages or three stages, a spatial feature amount extraction unit that extracts the spatial feature amount of the face image acquired by the face image acquisition unit, a blood pressure stage determination unit that determines the blood pressure stage of the subject based on the spatial feature amount extracted by the spatial feature amount extraction unit and the determination spatial feature amount, and an estimated blood pressure stage output unit that outputs the determination result by the blood pressure stage determination unit as an estimation result of the blood pressure stage of the subject.

The blood pressure estimation system extracts a spatial feature amount of the face image of the subject, determines a blood pressure stage of the subject based on the extracted spatial feature amount and the determination spatial feature amount, and outputs the determination result as an estimation result of the blood pressure stage of the subject.

A blood pressure estimation system is characterized in that the determination spatial feature amount stored in the determination feature amount storage unit is a spatial feature amount extracted by a machine learning unit, and the machine learning unit includes a learning data storage unit that stores a plurality of learning face images labeled corresponding to blood pressure stages consisting of the two stages or the three stages, respectively, a feature amount extraction unit that extracts the spatial feature amount of the learning face image using a learned model, and a feature amount learning unit that changes network parameters of the learned model based on a relationship between the extraction result obtained by the feature amount extraction unit and the label attached to the learning face image serving as an extraction target thereof such that the extraction accuracy of the spatial feature amount by the feature amount extraction unit becomes high.

This blood pressure estimation system uses the spatial feature amount extracted by the machine learning unit as a determination spatial feature amount. The machine learning unit stores a plurality of learning face images labeled corresponding to blood pressure stages consisting of two stages or three stages, extracts the spatial feature amount of the face image of the subject from the learning face image using the learned model, and changes network parameters of the learned model based on the relationship between the extraction result and the label attached to the learning face image serving as the extraction target thereof such that the extraction accuracy of the spatial feature amount of the face image of the subject becomes high.

The blood pressure estimation system is characterized in that

the face image is a face thermal image or a face visible image.

The blood pressure estimation system extracts the spatial feature amount of the face thermal image or the face visible image of the subject, determines a blood pressure stage of the subject based on the extracted spatial feature amount and the determination spatial feature amount, and outputs the determination result as the estimation result of the blood pressure stage of the subject.

A learning device is characterized by including comprising a learning data storage unit that stores a plurality of learning face images labeled corresponding to blood pressure stages consisting of two stages or three stages, a feature amount extraction unit that extracts a spatial feature amount of the learning face image using a learned model, and a feature amount learning unit that changes network parameters of the learned model based on a relationship between an extraction result obtained by the feature amount extraction unit and the label attached to the learning face image serving as an extraction target thereof such that extraction accuracy of the spatial feature amount by the feature amount extraction unit becomes high.

The learning device stores a plurality of learning face images labeled corresponding to blood pressure stages consisting of two stages or three stages, extracts, from the leaning face image, the spatial feature amount of the face image of the subject by using a learned model, and changes the network parameters of the learned model based on the relationship between the extraction result and the label attached to the learning face image serving as an extraction target thereof such that extraction accuracy of the spatial feature amount of the face image of the subject becomes high.

A blood pressure estimation method is characterized by including a face image acquisition step of acquiring a face image of a subject, and a blood pressure estimation step of estimating a blood pressure of the subject based on a spatial feature amount of the face image.

The blood pressure estimation method acquires the face image of the subject and estimates the blood pressure of the subject based on the spatial feature amount of the face image.

A blood pressure estimation method is characterized in that the blood pressure estimation step includes a correlation data storage step of storing correlation data showing a relationship between a weighted time series of an independent components of the face image and a blood pressure, a spatial feature amount extraction step of extracting the weighted time series of the independent components of the face image as the spatial feature amount by analyzing the independent components of the face image of the subject, a blood pressure determination step of determining the blood pressure value corresponding to the weighted time series extracted in the spatial feature amount extraction step on the basis of the correlation data, and an estimated blood pressure value output step of outputting a determination result obtained by the blood pressure determination step as an estimated value of the blood pressure of the subject.

In this blood pressure estimation method, the weighted time series of the independent components of the face image by analyzing the independent components of the face image of the subject, a blood pressure value corresponding to the extracted weighted time series is determined based on the correlation data, and the value is output as the estimated value of the blood pressure of the subject.

A blood pressure estimation method is characterized in that the blood pressure estimation step includes a correlation data storage step of storing correlation data showing the weight time series of the independent components of the face image and a relationship between a differential value thereof and a blood pressure, a weighted time series calculation step of calculating a weighted time series of the independent components of the face image as the spatial feature amount by analyzing the independent components of the face image acquired in the face image acquisition step, a weighted time series differential value calculation step of calculating a differential value of the weighted time series calculated by the weighted time series calculation step, a blood pressure determination step of determining, from the correlation data, a blood pressure value corresponding to the weighted time series calculated by the weighted time series calculation step and the differential value of the weighted time series calculated by the weighted time series differential value calculation unit; and an estimated blood pressure value output step of outputting the value determined by the blood pressure determination step as an estimated value of the blood pressure of the subject.

In the blood pressure estimation method, the weighted time series of the independent components of the face image is calculated by analyzing the independent components of the face image of the subject, and a differential value of the weighted time series is calculated. A blood pressure value corresponding to the weighted time series and the differential value are determined from the correlation data, and the value is output as an estimated value of the blood pressure of the subject.

A blood pressure estimation method is characterized in that the differential value of the weighted time series includes the first-order differential value and the second-order differential value of the weighted time series, and the weighted time series differential value calculation unit calculates a first-order differential value and a second-order differential value of the weighted time series.

In the blood pressure estimation method, the weighted time series and a blood pressure value corresponding to the first-order differential value and the second-order differential value thereof are determined from the correlation data, and the value is output as an estimated value of the blood pressure of the subject.

The blood pressure estimation method is characterized in that the blood pressure estimation step includes a determination feature amount storage step of storing a determination spatial feature amount corresponding to blood pressure stages consisting of two stages or three stages, a blood pressure stage determination step of determining a blood pressure stage of the subject based on the spatial feature amount of the face image of the subject and the spatial feature amount for the determination, and an estimated blood pressure stage output step of outputting a determination result obtained by the blood pressure stage determination step as an estimation result of the blood pressure stage of the subject.

In the blood pressure estimation method, a spatial feature amount of the face image of the subject is extracted, a blood pressure stage of the subject is determined based on the extracted spatial feature amount and the determination spatial feature amount, and the determination result is output as an estimation result of the blood pressure stage of the subject is output.

A learning method is characterized by including a learning data storage step of storing a plurality of learning face images labeled corresponding to blood pressure stages consisting of two stages or three stages, a feature amount extraction step of extracting a spatial feature amount of the learning face image using a learned model, and a feature amount learning step of changing network parameters of the learned model such that extraction accuracy of the spatial feature amount by the feature amount extraction step based on a relationship between the extraction result obtained by the feature amount extraction step and the label attached to the learning face image serving as an extraction target thereof.

In the learning method, a plurality of leaning face images labeled corresponding to blood pressure stages consisting of two stages or three stages are stored, a spatial feature amount of the face image of the subject is extracted from the learning face image using the learned model, and network parameters of the learned model are changed on the basis of a relationship between the extraction result and a label attached to the learning face image serving as an extraction target thereof such that extraction accuracy of the spatial feature amount of the face image of the subject becomes high.

A program which is a non-volatile recording medium recording a program for causing a computer to function as a means for estimating a blood pressure of a subject, is characterized by including a correlation data storage step of storing correlation data showing a relationship between a weighted time series of independent components of a face image and a blood pressure, a face image acquisition step of acquiring the face image of the subject, a spatial feature amount extraction unit that extracts the weighted time series of the independent components of the face image as a spatial feature amount of the face image by analyzing the independent components of the face image acquired in the face image acquisition step, and an estimated blood pressure value output step of calculating a blood pressure value corresponding to the weighted time series extracted by the spatial feature amount extraction step from the correlation data to output the value as an estimated value of the blood pressure of the subject.

The program is installed in one computer or a plurality of computers working in cooperation with each other, is executed, and causes a system consisting of the computer or the plurality of computers to function as a means that extracts the weighted time series of the independent components of the face image by analyzing the independent components of the face image of the subject, determines a value of a blood pressure corresponding to the extracted weighted time series from the correlation data, and outputs the value as an estimated value of the blood pressure of the subject.

A program which is a non-volatile recording medium recording a program for causing a computer to function as a means for estimating a blood pressure of a subject, is characterized by including a correlation data storage step of storing correlation data showing the relationship between blood pressure and the weighted time series of the independent component of the face image and its differential value; a weighted time series calculation step of calculating a weighted time series of the independent components of the face image as the spatial feature amount by analyzing the independent components of the face image acquired in the face image acquisition step, a weighted time series differential value calculation step of calculating a differential value of the weighted time series calculated by the weighted time series calculation step, a blood pressure determination step of determining, from the correlation data, a blood pressure value corresponding to the weighted time series calculated by the weighted time series calculation step and the differential value of the weighted time series calculated by the weighted time series differential value calculation unit, and an estimated blood pressure value output step of outputting the value determined by the blood pressure determination step as an estimated value of the blood pressure of the subject.

The program is installed in one computer or a plurality of computers working in cooperation with each other, is executed, and causes a system consisting of the computer or the plurality of computers to function as a means that calculates a weighted time series of independent components of the face image by analyzing the independent components of the face image of the subject, calculates a differential value of the weighted time series, determines the weighted time series and a blood pressure value corresponding to the differential value thereof from the correlation data, and outputs the value as an estimated value of the blood pressure of the subject.

A program is characterized in that the differential value of the weighted time series includes a first-order differential value and a second-order differential value of the weighted time series, and the weighted time series differential value calculation step is a step of calculating the first-order differential value and the second-order differential value of the weighted time series.

The program is installed in one computer or a plurality of computers working in cooperation with each other, is executed, and causes the computer or the plurality of computers to function as a means that determines the weighted time series and a blood pressure value corresponding to the first-order differential value and the second-order differential value from the correlation data, and outputs the value as an estimated value of the blood pressure of the subject.

A program which is a non-volatile recording medium recording a program for causing a computer to function as a means for estimating a blood pressure of a subject, is characterized by including a determination feature amount storage step of storing a determination spatial feature amount corresponding to a blood pressure stage consisting of two stages or three stages, a face image acquisition step of acquiring a face image of the subject, a blood pressure stage determination step of determining a blood pressure stage of the subject based on the face image acquired in the face image acquisition step and the determination spatial feature amount; and an estimated blood pressure stage output step of outputting a determination result obtained by the blood pressure stage determination step as an estimation result of the blood pressure stage of the subject.

The program is installed in one computer or a plurality of computers working in cooperation with each other, is executed, and causes a system consisting of the computer or the plurality of computers to function as a means that extracts a spatial feature amount of the face image of the subject, determines a blood pressure stage of the subject based on the extracted spatial feature amount and a determination spatial feature amount, and outputs the determination result as an estimated result of the blood pressure stage of the subject.

A program is characterized by including a learning data storage step of storing a plurality of learning face images labeled corresponding to blood pressure stages consisting of two steps or three steps, a feature amount extraction step of extracting a spatial feature amount of the face image from the learning face image using a learned model, and a learning step of changing network parameters of the learned model based on a relationship between an extraction result obtained by the feature amount extraction step and a label attached to the learning face image serving as an extraction target thereof such that extraction accuracy of the spatial feature amount obtained by the feature amount extraction step becomes high, wherein the determination feature amount storage step is a step of storing the spatial feature amount extracted by the feature amount extraction step.

The program is installed in one computer or a plurality of computers working in cooperation with each other, is executed, and causes a system consisting of the computer or the plurality of computers to function as a means that stores a plurality of learning face images labeled corresponding to blood pressure stages consisting of two stages or three stages, extracts a spatial feature amount of the learning face image using the learned model, changes network parameters of the learned model based on a relationship between the extraction result and a label attached to the learning face image serving as an extraction target thereof such that extraction accuracy of the spatial feature amount becomes high, and stores the extracted spatial feature amount.

A program which is a non-volatile recording medium recording a program for causing a computer to function as a learning device for estimating a blood pressure of a subject, is characterized by including a learning data storage step of storing a plurality of learning face images labeled corresponding to blood pressure stages consisting of two steps or three steps, a feature amount extraction step of extracting a spatial feature amount of the learning face image using a learned model, and a feature amount learning step of changing network parameters of the learned model based on a relationship between an extraction result obtained by the feature amount extraction step and a label attached to the learning face image serving as an extraction target thereof such that extraction accuracy of the spatial feature amount obtained by the feature amount extraction step becomes high.

The program is installed in one computer or a plurality of computers working in cooperation with each other, is executed, and causes a system consisting of the computer or the plurality of computers to function as a means that stores a plurality of learning face images labeled corresponding to blood pressure stages consisting of two stages or three stages, extracts a spatial feature amount of the face image of the subject from the learning face image by using the learned model, and changes network parameters of the learned model based on a relationship between the extraction result and a label attached to the learning face image serving as an extraction target thereof such that extraction accuracy of the spatial feature amount of the face image of the subject becomes high.

### Advantages

According to the blood pressure estimation system a blood pressure of the subject is estimated based on a spatial feature amount of the face image of the subject acquired in a non-contact manner to make it possible to instantaneously estimate the blood pressure of the subject in a non-contact manner.

More specifically, for example, in the conventional technique described in Patent Literature 1, since a blood pressure is estimated based on information of time change in luminance of a skin image of a subject, that is, a temporal feature amount of the skin image and information of temporal change in distance between the subject and a receiving antenna, that is, a temporal feature amount of a skin position, a time required to extract the temporal feature amount of the skin image and the temporal feature amount of the skin position is necessary every blood pressure estimation, and the blood pressure of the subject cannot be instantaneously estimated. However, in the invention described above is configured such that the blood pressure of the subject is estimated based on a spatial feature amount of the face image of the subject acquired in a non-contact manner, the blood pressure of the subject can be estimated by only information processing of the spatial feature amount of the face image. Thus, a time required to estimate the temporal feature amount of the skin image and the temporal feature amount of the skin position is not necessary every blood pressure estimation unlike in the conventional technique, and, the face image of the subject is acquired, the blood pressure can be instantaneously and accurately estimated.

Furthermore, for example, in the conventional technique described in Patent Literature 1, since, in addition to an imaging means for acquiring a skin image of a subject, a calculation means for calculating pulse wave timing, a distance measuring means including a receiving antenna for measuring a distance to the skin of the subject, and a calculation means for calculating a heartbeat timing are required, a system configuration is large in scale. However, in the invention described above, since a blood pressure estimation system is configured by a face image acquisition unit for acquiring a face image of a subject in a non-contact manner and a blood pressure estimation unit for estimating a blood pressure of the subject based on a spatial feature amount of the face image, a simple system configuration can be achieved, and costs required for the system configuration can also be reduced.

According to the blood pressure estimation system based on the weighted time series of the independent components of the face image of the subject, the blood pressure of the subject can be accurately and instantaneously estimated in a non-contact manner.

According to the blood pressure estimation system based on the weighted time series of the independent components of the face image of the subject, the blood pressure of the subject can be instantaneously and accurately estimated in a non-contact manner. In addition, according to the blood pressure estimation system, since a subsequent change rate of the spatial feature amount can be estimated based on a differential value of the weighted time series of the independent components of the face image of the subject, a change in blood pressure after the measurement can be accurately predicted.

According to the blood pressure estimation system since the subsequent change rate of the spatial feature amount can be estimated based on the first-order differential value and the second-order differential value of the weighted time series, prediction and estimation of the subsequent blood pressure change of the substance can be accurately performed without a significant increase in calculation amount. That is, since the change rate of the blood pressure can be precisely analyzed, a change in blood pressure of the subject after the measurement can be accurately predicted.

According to the blood pressure estimation system based on independent components of a face thermal image or a face visible image of the subject, the blood pressure of the subject can be instantaneously and accurately estimated in a non-contact manner.

According to the blood pressure estimation system the blood pressure stage of the subject can be instantaneously and accurately estimated in a non-contact manner based on the spatial feature amount of the face image of the subject.

According to the blood pressure estimation system since the network parameters of the learned model can be changed so that the extraction accuracy of the spatial feature amount of the face image of the subject becomes high, the blood pressure stage of the subject can be instantaneously and highly accurately estimated in a non-contact manner based on the spatial feature amount of the face image of the subject.

According to the blood pressure estimation system the blood pressure stage of the subject can be instantaneously and highly accurately estimated in a non-contact manner based on the spatial feature amount of the face thermal image or the face visible image of the subject.

According to the learning device the network parameters of the learned model can be changed so that the extraction accuracy of the spatial feature amount of the face image of the subject becomes high. As a result, the blood pressure stage of the subject can be instantaneously and highly accurately estimated in a non-contact manner.

According to the blood pressure estimation method the blood pressure of the subject is estimated based on the spatial feature amount of the face image of the subject, so that the blood pressure of the subject can be instantaneously and accurately in a non-contact manner.

According to the blood pressure estimation method the blood pressure of the subject can be instantaneously and accurately estimated in a non-contact manner based on the weighted time series of the independent components of the face image of the subject.

According to the blood pressure estimation method the blood pressure of the subject can be instantaneously and accurately estimated in a non-contact manner based on the weighted time series of the independent components of the face image of the subject. Furthermore, according to this blood pressure estimation method, a subsequent change rate of the spatial feature amount can be estimated based on the differential value of the weighted time series of the independent components of the face image of the subject, so that the subsequent change in blood pressure of the subject can be predicted and estimated.

According to the blood pressure estimation method since the subsequent change rate of the spatial feature amount can be estimated based on the first-order differential value and the second-order differential value of the weighted time series, a subsequent change in blood pressure of the subject can be more accurately predicted and estimated. More specifically, since the change rate of the blood pressure can be accurately analyzed, a change in blood pressure after the measurement state can be accurately predicted.

According to the blood pressure estimation method the blood pressure stage of the subject can be instantaneously estimated in a non-contact manner based on the spatial feature amount of the face image of the subject.

According to the learning method the network parameters of the learned model can be changed so that the extraction accuracy of the spatial feature amount of the face image of the subject becomes high.

According to the program a blood pressure estimation system having a function that, by analyzing the independent components of the face image of the subject, extracts the weighted time series of the independent components of the face image, determines the value of the blood pressure corresponding to the extracted weighted time series from the correlation data, and outputs the value as an estimated value of the blood pressure of the subject can be achieved using one computer or a plurality of computers working in cooperation with each other.

According to the program a blood pressure estimation system having a function that, by analyzing the independent components of the face image of the subject, calculates the weighted time series of the independent components of the face image, calculates a differential value of the weighted time series, determines the weighted time series and the value of the blood pressure corresponding to the differential value thereof from the correlation data, and outputs the value as an estimated value of the blood pressure of the subject can be achieved using one computer or a plurality of computers working in cooperation with each other.

According to the program a blood pressure estimation system having a function that determines the weighted time series and a blood pressure value corresponding to the first-order differential value and the second-order differential value thereof from the correlation data and outputs the value as an estimated value of the blood pressure of the subject can be achieved using one computer or a plurality of computers working in cooperation with each other.

According to the program a blood pressure estimation system having a function that extracts the spatial feature amount of the face image of the subject, determines the blood pressure stage of the subject based on the extracted spatial feature amount and the determination spatial feature amount, and outputs the determination result as an estimation result of the blood pressure stage of the subject can be achieved using one computer or a plurality of computers working in cooperation with each other.

According to the program a plurality of learning face images labeled corresponding to each of two or three blood pressure stages are stored, and the spatial feature amount of the learning face image is extracted using the learned model, based on the relationship between the extraction result and the label attached to the learning face image that is the extraction target of the learned model so that the extraction accuracy of the spatial feature amount becomes high. The blood pressure estimation system having a function of changing the network parameter of the learned model and storing the extracted spatial feature amount can be realized by using one or a plurality of computers working in cooperation with each other.

According to the program a learning device having a function that stores a plurality of learning face images labeled corresponding to blood pressure stages consisting of two stages or three stages, extracts a spatial feature amount of the face image of the subject from the learning face images using the learned model, and changes network parameters of the learned model based on a relationship between the extraction result and the label attached to the learning face image serving as the extraction target such that the extraction accuracy of the spatial feature amount of the face image of the subject becomes high can be achieved by one computer or a plurality of computers working in cooperation with each other.

### BRIEF DESCRIPTION OF THE DRAWINGS

FIG. 1 It is a block diagram of a blood pressure estimation system according to a first embodiment of the present invention.

FIG. 2 is a flow chart showing processing contents of the blood pressure estimation system in FIG. 1.

FIG. 3 is a flow chart of a blood pressure estimation process in FIG. 2.

FIG. 4 is a block diagram of a blood pressure estimation system according to a second embodiment of the present invention.

FIG. 5 is a flowchart showing processing contents of the blood pressure estimation system in FIG. 4.

FIG. 6 is flow chart of the blood pressure estimation process in FIG. 4.

FIG. 7 is a block diagram of a blood pressure estimation system according to the second embodiment of the present invention.

FIG. 8 is a flow chart showing processing contents of the blood pressure estimation system in FIG. 7.

FIG. 9 is a flow chart of the blood pressure estimation process in FIG. 7.

FIG. 10 is a flow chart of a learning process in the blood pressure estimation system according to the second embodiment.

FIG. 11 is a diagram showing an experimental protocol.

FIG. 12 is a diagram exemplifying an extracted face thermal image (FTI).

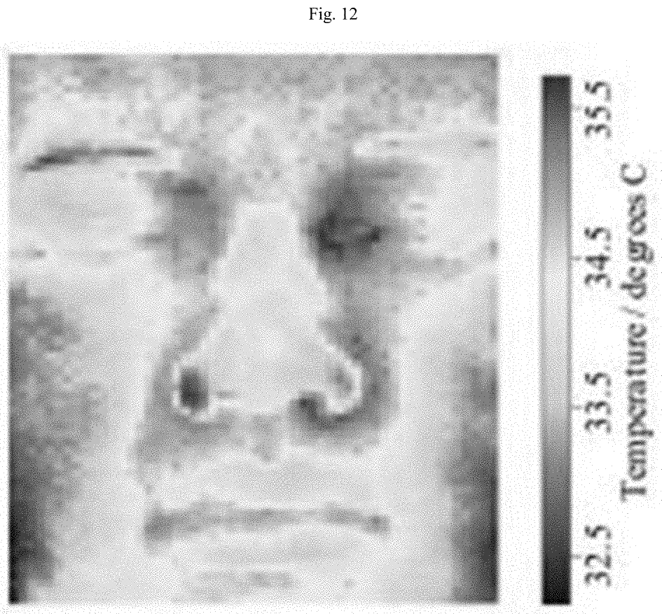

FIG. 13 is diagram exemplifying time series changes of a mean blood pressure (MBP), a cardiac output (CO), a total peripheral vascular resistance (TPR), and a heart rate (HR).

FIG. 14 is diagram exemplifying independent components and a weighted time series extracted from an observation signal.

FIG. 15 is a diagram exemplifying an estimated value and a measurement result of the mean blood pressure.

FIG. 16 is a diagram exemplifying an extracted independent component image and a corresponding weight time series.

FIG. 17 is a diagram exemplifying a derivation result of an individual model.

FIG. 18 is a diagram exemplifying a derivation result of a general model.

FIG. 19 is diagram exemplifying a time series of a mean blood pressure and a blood pressure stage in a blood pressure change experiment (subject G).

FIG. 20 is diagram exemplifying a mean blood pressure displacement in the blood pressure change experiment.

FIGS. 21(A) and 21(B) are diagrams showing a representative example of a confusion matrix (subject A) in blood pressure stage estimation. FIG. 21A is a confusion matrix of two-stage estimation, and FIG. 21B is a confusion matrix of three-stage estimation.

FIGS. 22(A) and 22(B) are diagrams showing a representative example of a confusion matrix (subject G) in blood pressure stage estimation. FIG. 22A is a confusion matrix of two-stage estimation, and FIG. 22B is a confusion matrix of three-stage estimation.

FIGS. 23(A) and 23(B) are diagrams exemplifying a relationship between a difference between the highest and lowest values of the mean blood pressure in the blood pressure change experiment and a correct answer rate of blood pressure stage estimation. FIG. 23A is a case of the two-stage estimation, and FIG. 23B is a case of the three-stage estimation.

FIGS. 24(A), 24(B) and 24(C) are diagrams showing a feature map (subject A) in a first convolutional layer. FIG. 24A shows a case of High Level, FIG. 24B shows a case of Middle Level, and FIG. 24C shows a case of Low Level.

FIGS. 25(A), 25(B) and 25(C) are diagrams showing a feature map (subject A) in a second convolutional layer. FIG. 25A shows a case of High Level, FIG. 25B shows a case of Middle Level, and FIG. 25C shows a case of Low Level.

FIGS. 26(A), 26(B) and 26(C) are diagrams showing a feature map (subject A) in a third convolutional layer. FIG. 26A shows a case of High Level, FIG. 26B shows a case of Middle Level, and FIG. 26C shows a case of Low Level.

FIGS. 27(A), 27(B) and 27(C) are diagrams showing a feature map (subject G) in the first convolutional layer. FIG. 27A shows a case of High Level, FIG. 27B shows a case of Middle Level, and FIG. 27C shows a case of Low Level.

FIGS. 28(A), 28(B) and 28(C) are diagrams showing a feature map (subject G) in the second convolutional layer. FIG. 28A shows a case of High Level, FIG. 28B shows a case of Middle Level, and FIG. 28C shows a case of Low Level.

FIGS. 29(A), 29(B) and 29(C) are diagrams showing a feature map (subject G) in the third convolutional layer. FIG. 29A shows a case of High Level, FIG. 29B shows a case of Middle Level, and FIG. 29C shows a case of Low Level.

### BEST MODE FOR CARRYING OUT THE INVENTION

Embodiments of the present invention will be described below with reference to the accompanying drawings.

### First Embodiment

### [Configuration]

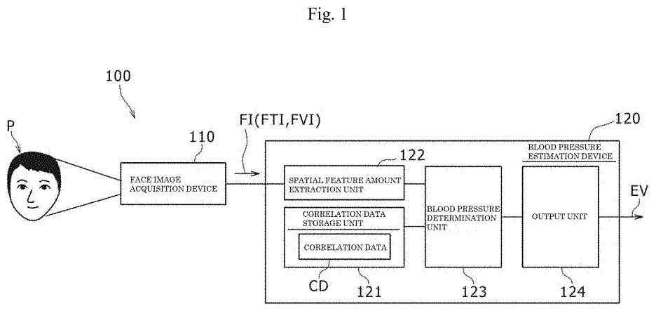

A blood pressure estimation system 100 according to a first embodiment shown in FIG. 1 includes a face image acquisition device (face image acquisition unit) 110 and a blood pressure estimation device (blood pressure estimation unit) 120.

The face image acquisition device 110 is a device for capturing a face image FI of a subject P. The face image FI may be a face thermal image FTI or a face visible image FVI. When the face image FI is the face thermal image FTI, an infrared thermography is used as the face image acquisition device 110. When the face image FI is the face visible image FVI, the visible image capturing device is used as the face image acquisition device 110.

A blood pressure estimation device 120 is realized by installing and executing a program according to the present invention in a general-purpose computer.

The blood pressure estimation device 120 is a device that uses a weighted time series of independent components of the face image FI as a spatial feature amount of the face image FI and estimates a blood pressure of the subject P based on the face image FI acquired by the face image acquisition device 110 and correlation data CD representing a relationship between a weighted time series A of the independent components of the face image FI and a blood pressure.

The blood pressure estimation device 120 includes a correlation data storage unit 121, a spatial feature amount extraction unit 122, a blood pressure determination unit 123, and an estimated blood pressure value output unit 124.

The correlation data storage unit 121 is a functional block that stores the correlation data CD.

The spatial feature amount extraction unit 122 is a functional block extracts the weighted time series of the independent components of the face image FI as the spatial feature amount by analyzing the independent components of the face image FI acquired by the face image acquisition device 110.

The blood pressure determination unit 123 is a functional block that determines a blood pressure value corresponding to the weighted time series A extracted by the spatial feature amount extraction unit 122 from the correlation data CD.

The estimated blood pressure value output unit 124 is a functional block that outputs the value determined by the blood pressure determination unit 123 as an estimated value EV of the blood pressure of the subject P.

### [Action]

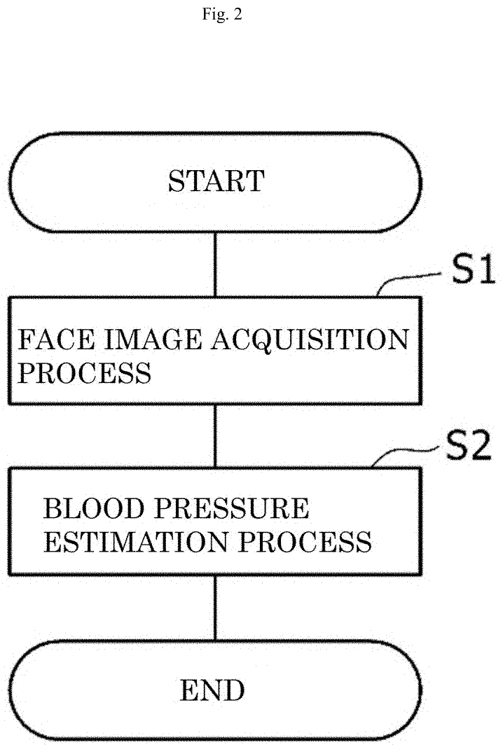

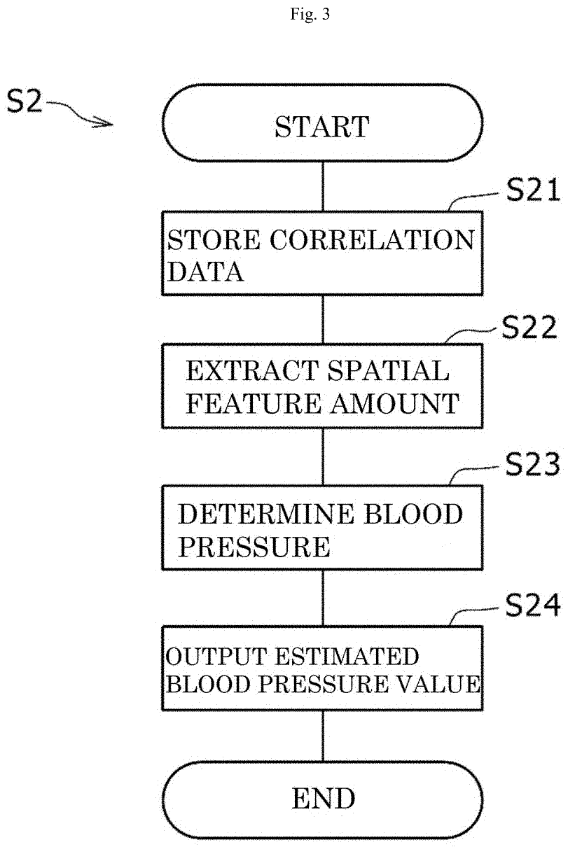

The flow of processing in the blood pressure estimation system 100 configured as above will be described with reference to the flowcharts of FIGS. 2 and 3.

As shown in FIG. 2, the blood pressure estimation system 100 executes a face image acquisition process S1 and a blood pressure estimation process S2.

The face image acquisition process S1 is a process of acquiring the face image FI of the subject P.

The blood pressure estimation process S2 is a process of estimating the blood pressure of the subject P based on the spatial feature amount of the face image FI of the subject P acquired by the face image acquisition process S1.

As shown in FIG. 3, the blood pressure estimation process S2 includes a correlation data storage process S21, a spatial feature amount extraction process S22, a blood pressure determination process S23, and an estimated blood pressure value output process S24.

The correlation data storage process S21 is a process of storing the correlation data CD.

The spatial feature amount extraction process S22 is a process of extracting the weighted time series of the independent components of the face image FI as the spatial feature amount of the face image FI by analyzing the independent components of the face image FI of the subject P acquired by the face image acquisition process S1.

The blood pressure determination process S23 is a process of determining the blood pressure value corresponding to the weighted time series A extracted by the spatial feature amount extraction process S22 from the correlation data CD.

The estimated blood pressure value output process S24 is a process of outputting a determination result obtained by the blood pressure determination process S23 as an estimated value EV of the blood pressure of the subject P.

### [Operation/Advantage]

In the blood pressure estimation system 100 configured as described above, the face image FI of the subject P is captured by the face image acquisition device 110. The captured face image FI is input to the blood pressure estimation device 120. The blood pressure estimation device 120 extracts the weighted time series A of the independent components of the face image FI by analyzing the independent components of the face image FI of the subject P, determines the blood pressure value corresponding to the extracted weighted time series A from the correlation data CD, and outputs the value as the estimated value EV of the blood pressure of the subject P.

According to the blood pressure estimation system 100, the blood pressure of the subject P can be instantaneously and accurately estimated in a non-contact manner based on the weighted time series A of the independent components of the face image FI of the subject P. More specifically, one face image FI of the subject P is only captured to make it possible to instantaneously and accurately the blood pressure of the subject P.

### Second Embodiment

### [Configuration]

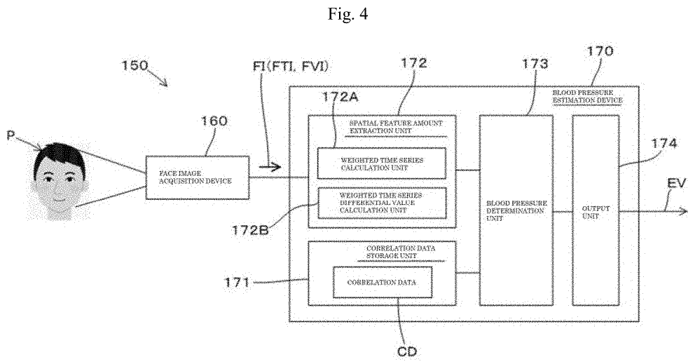

A blood pressure estimation system 150 according to a second embodiment shown in FIG. 4 includes a face image acquisition device (face image acquisition unit) 160 and a blood pressure estimation device (blood pressure estimation unit) 170.

The face image acquisition device 160 is a device for capturing the face image FI of the subject P. The face image FI may be the face thermal image FTI or the face visible image FVI. When the face image FI is the face thermal image FTI, an infrared thermography is used as the face image acquisition device 160. When the face image FI is the face visible image FVI, a visible image capturing device is used as the face image acquisition device 160.

The face image acquisition device 160 captures the three face images FI with one shot. A time for one shot is, for example, 2 seconds. Hereinafter, the first image is called a “first image FI1”, the second image is called a “second image FI2”, and the third image is called a “third image FI3”, hereinafter.

The blood pressure estimation device 170 is realized by installing and executing the program according to the present invention in a general-purpose computer.

The blood pressure estimation device 170 is a device that uses a weighted time series A of the independent components of the face image FI, a first-order differential value A′ of the weighted time series A, and the second-order differential value A″ of the weighted time series A as the spatial feature amounts of the face image FI and estimate the blood pressure of the subject P based on the correlation data CD representing a relationship between the values A, A′, and A″ and the blood pressure.

The blood pressure estimation device 170 has a correlation data storage unit 171, a spatial feature amount extraction unit 172, a blood pressure determination unit 173, and an estimated blood pressure value output unit 174.

The correlation data storage unit 171 is a functional block that stores the correlation data CD.

The spatial feature amount extraction unit 172 has a weighted time series calculation unit 172A and a weighted time series differential value calculation unit 172B.

The weighted time series calculation unit 172A is a functional block that calculates the weighted time series A of the independent components of the first image FI1, the second image FI2, and the third image FI3 as spatial feature amounts by analyzing the independent components of the three face images FI of one shot acquired by the face image acquisition device 160, i.e., the first image FI1, the second image FI2, and the third image FI3. Hereinafter, the weighting time series of the first image FI1 is called a “first weighting time series A1”, the weighting time series of the second image FI2 is called a “second weighting time series A2”, and the weighting time series of the third image FI3 is called a “third weighted time series A3”.

The weighted time series differential value calculation unit 172B is a functional block that calculates the first-order differential value A′ and the second-order differential value A″ from the first weighted time series A1, the second weighted time series A2, and the third weighted time series A3 calculated by the weighted time series calculation unit 172A.

In this example, the weighted time series differential value calculation unit 172B calculates a difference between the first weighted time series A1 and the second weighted time series A2 to calculate the first weighted time series first-order differential value A1′ and calculates a difference between the second weighted time series A2 and the third weighted time series A3 to calculate the second weighted time series first-order differential value A2′. The weighted time series differential value calculation unit 172B calculates a difference between the first weighted time series first-order differential value A1′ and the second weighted time series first-order differential value A2′ to calculate the weighted time series second-order differential value A″.

The blood pressure determination unit 173 is a functional block that determines blood pressure values corresponding to the weighted time series A calculated by the weighted time series calculation unit 172A, the first weighted time series first-order differential value A1′, the second weighted time series first-order differential value A2′, and the weighted time series second-order differential value A″ based on the correlation data CD. In this example, the blood pressure determination unit 173 defines a mean value between the first weighted time series first-order differential value A1′ and the second weighted time series first-order differential value A2′ as the weighted time series first-order differential value A′, and determines the blood pressure values corresponding to the weighted time series A, the weighted time series first-order differential value A′, and the weighted time series second-order differential value A″ from the correlation data CD.

The estimated blood pressure value output unit 174 is a functional block that outputs a value determined by the blood pressure determination unit 173 as the estimated value EV of the blood pressure of the subject P.

### [Action]

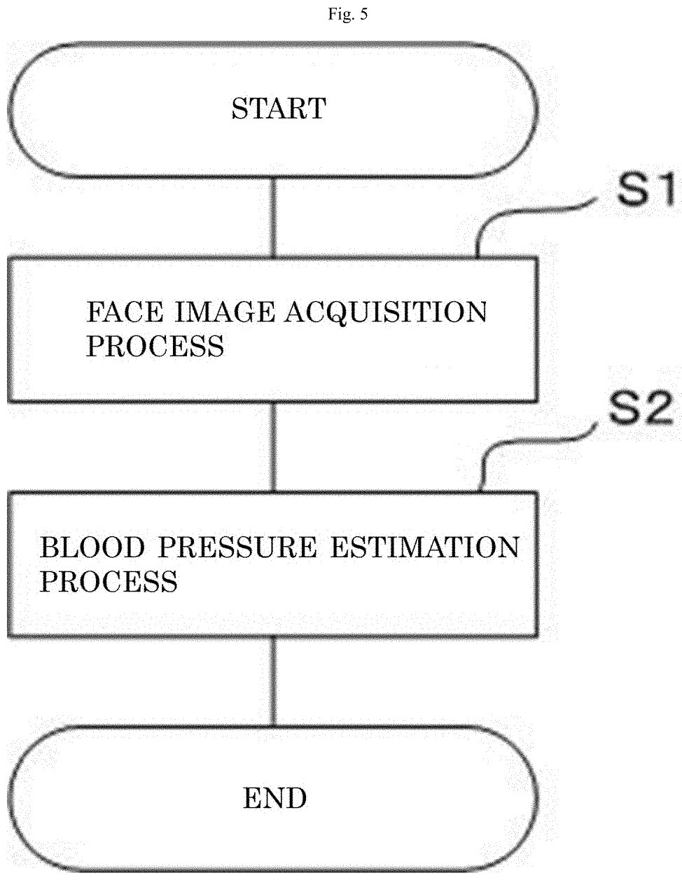

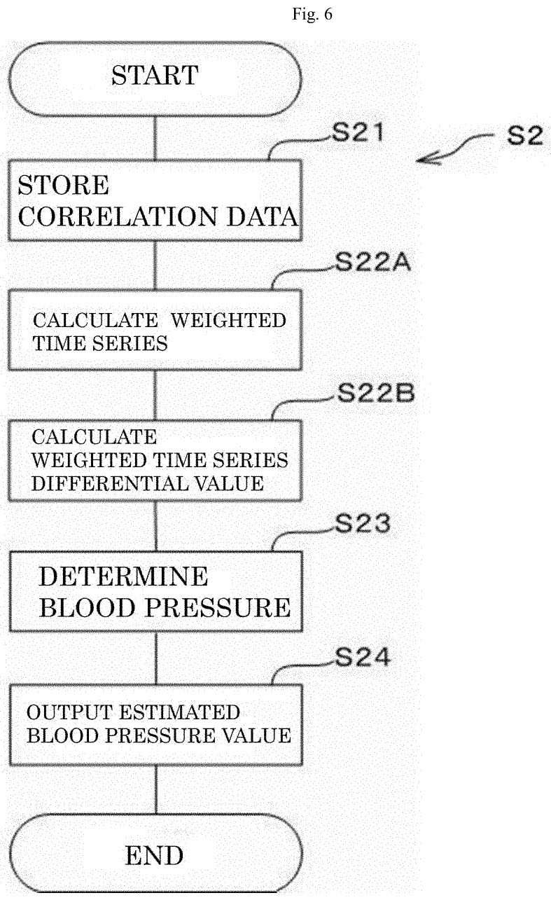

A flow of processes in the blood pressure estimation system 150 configured as described above will be described below in accordance with the flow charts in FIG. 5 and FIG. 6.

As shown in FIG. 5, the blood pressure estimation system 150 executes a face image acquisition process S1 and a blood pressure estimation process S2.

The face image acquisition process S1 is a process of acquiring the face image FI of the subject P.

The blood pressure estimation process S2 is a process of estimating the blood pressure of the subject P based on the spatial feature amount of the face image FI of the subject P acquired by the face image acquisition process S1.

As shown in FIG. 6, the blood pressure estimation process S2 includes a correlation data storage process S21, a weighted time series calculation process S22A, a weighted time series differential value calculation process S22B, a blood pressure determination process S23, and an estimated blood pressure value output process S24.

The correlation data storage process S21 is a process of storing the correlation data CD.

The weighted time series calculation process 22A is a process of calculating the weighted time series A of the independent components of the face image FI as the spatial feature amount of the face image FI by analyzing the independent components of the face image FI of the subject P acquired by the face image acquisition process S1.

The weighted time series differential value calculation process 22B is a process of calculating differential values of the weighted time series A calculated by the weighted time series calculation process 22A, i.e., the first weighted time series first-order differential value A1′, the second weighted time series first-order differential value A2′, and the weighted time series second-order differential value A″.

The blood pressure determination process S23 is a process of determining the blood pressure values corresponding to the weighted time series A, the weighted time series first-order differential value A′, and the weighted time series second-order differential value A″ based on the correlation data CD.

The estimated blood pressure value output process S24 is a process of outputting the determination result obtained by the blood pressure determination process S23 as the estimated value EV of the blood pressure of the subject P.

### [Operation/Advantages]

In the blood pressure estimation system 150 configured as described above, the face image FI of the subject P is captured by the face image acquisition device 160. The imaged face image FI is input to the blood pressure estimation device 170. The blood pressure estimation device 170 extracts the weighted time series A of the independent components of the face image FI by analyzing the independent components of the face image FI of the subject P, further calculates the first-order differential value A′ and the second-order differential value A″ thereof, determines the blood pressure values corresponding to the values A, A′, and A″ based on the correlation data CD, and outputs the value as the estimated value EV of the blood pressure of the subject P.

According to the blood pressure estimation system 150, the blood pressure of the subject P can be instantaneously and accurately estimated in a non-contact manner based on the weighted time series A of the independent components of the face image FI of the subject P and the differential values A′ and A″ thereof. More specifically, when the three face image FI of the subject P is only captured with one shot, the blood pressure of the subject P can be instantaneously and accurately estimated.

Furthermore, according to the blood pressure estimation system 150, the rate of change of the spatial feature amount thereafter can be estimated based on the differential values A′ and A″ of the weighted time series A of the independent component of the face image FI of the subject P. Since it is possible to make an estimation, it is possible to make a predictive estimation of the change in blood pressure of the subject P thereafter. That is, since the rate of change in blood pressure can be accurately analyzed, it is possible to accurately predict the change in blood pressure after the measurement.

In the above example, the mean value of the first weighted time series first-order differential value A1′ and the second weighted time series first-order differential value A2′ is defined as the weighted time series first-order differential value A′. However, any one of the first weighted time series first-order differential value A1′ and the second weighted time series first-order differential value A2′ may be defined as the weighted time series first-order differential value A′.

### Third Embodiment

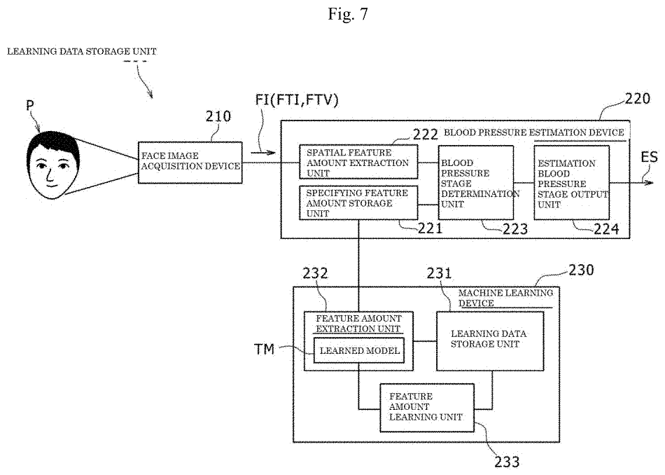

A blood pressure estimation system 200 according to the second embodiment shown in FIG. 7 includes a face image acquisition device (face image acquisition unit) 210, a blood pressure estimation device (blood pressure estimation unit) 220, and a learning device (machine learning unit) 230.

The face image acquisition device 210 is a device for capturing the face image FI of the subject P. The face image FI may be the face thermal image FTI or the face visible image FVI. When the face image FI is the face thermal image FTI, an infrared thermography is used as the face image acquisition device 210. When the face image FI is the face visible image FVI, a visible image capturing device is used as the face image acquisition device 210.

The blood pressure estimation device 220 is realized by installing and executing the program according to the present invention in a general-purpose computer.

The blood pressure estimation device 220 is a device that estimates the blood pressure of the subject P based on the spatial feature amount of the face image FI acquired by the face image acquisition device 210.

The blood pressure estimation device 220 includes a determination feature amount storage unit 221, a spatial feature amount extraction unit 222, a blood pressure stage determination unit 223, and an estimated blood pressure stage output unit 224.

The determination feature amount storage unit 221 is a functional block that stores a determination spatial feature amount corresponding to blood pressure stages consisting of two stages or three stages. The determination spatial feature amount is a spatial feature amount extracted by the learning device 230.

The spatial feature amount extraction unit 222 is a functional block that extracts a spatial feature amount of the face image FI acquired by the face image acquisition device 210.

The blood pressure stage determination unit 223 is a functional block that determines the blood pressure stage of the subject P based on the spatial feature amount extracted by the spatial feature amount extraction unit 222 and the determination spatial feature amount.

The estimated blood pressure stage output unit 224 is a functional block that outputs the determination result obtained by the blood pressure stage determination unit 223 as an estimation result ES of the blood pressure stage of the subject P.

The learning device 230 includes a learning data storage unit 231, a feature amount extraction unit 232, and a feature amount learning unit 233.

The learning data storage unit 231 is a functional block that stores a plurality of learning face images labeled corresponding to the blood pressure stages consisting of two stages or three stages, respectively.

The feature amount extraction unit 232 is a functional block that extracts the spatial feature amount of the learning face image using a learned model TM.

The feature amount learning unit 233 is a functional block that changes network parameters of the learned model TM based on a relationship between the extraction result obtained by the feature amount extraction unit 232 and the label attached to the learning face image serving as the extraction target thereof such that extraction accuracy of the spatial feature amount obtained by the feature amount extraction unit 232 becomes high. The learned model TM is generated such that machine learning of the spatial feature amount of the face image of the subject P included in the learning face image is performed using a plurality of learning face images labeled corresponding to the blood pressure stages consisting of two stages or three stages as teacher data.

### [Action]

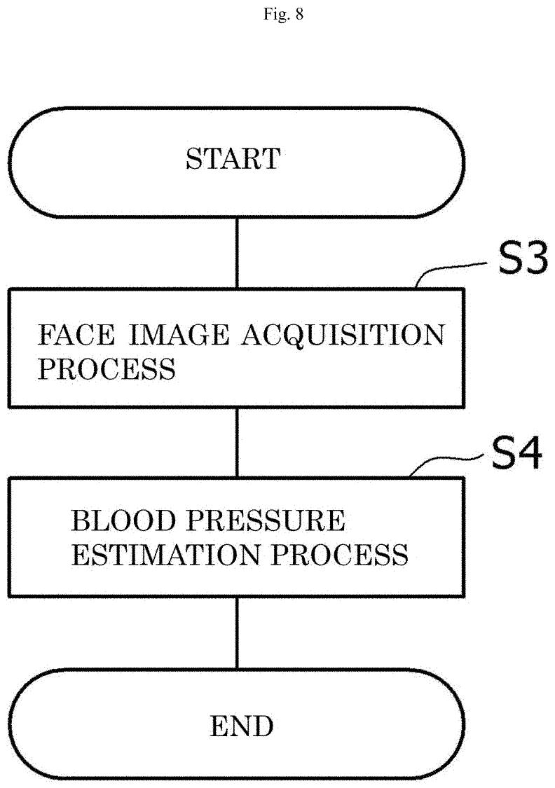

The flow of processes in the blood pressure estimation system 200 configured as described above will be described below with reference to the flowcharts in FIG. 8 to FIG. 10.

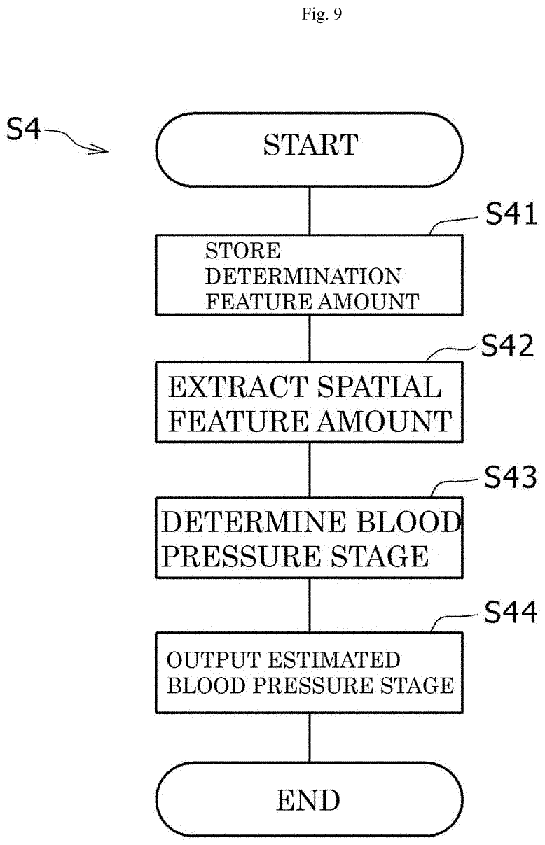

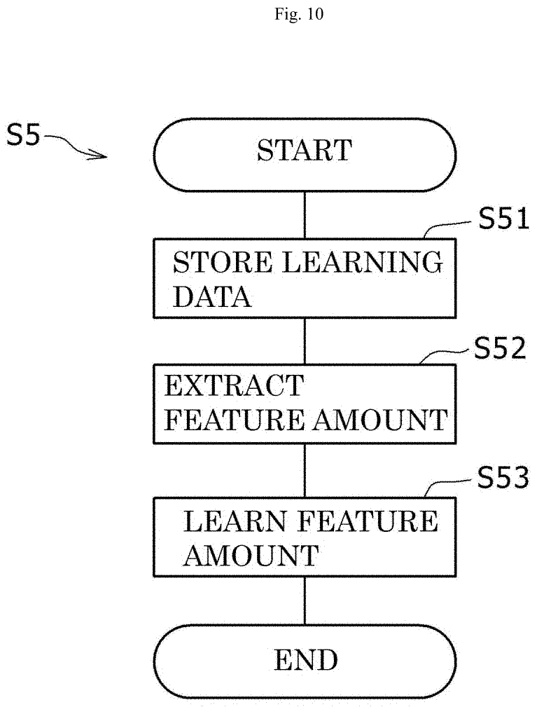

The blood pressure estimation system 200 executes a face image acquisition process S3 and a blood pressure estimation process S4 shown in FIG. 8 and FIG. 9, and a learning process S5 shown in FIG. 10.

The face image acquisition process S3 is a process of acquiring the face heat image FI of the subject P.

The blood pressure estimation process S4 is a process of estimating the blood pressure of the subject P based on a spatial feature amount of the face image FI acquired by the face image acquisition process S3.

As shown in FIG. 9, the blood pressure estimation process S4 includes a determination feature amount storage process S41, a spatial feature amount extraction process S42, a blood pressure stage determination process S43, and an estimated blood pressure stage output process S44.

The determination feature amount storage process S41 is a process of storing a determination spatial feature amount corresponding to blood pressure stages consisting of two stages or three stages.

The spatial feature amount extraction process S42 is a process of extracting the spatial feature amount of the face image FI acquired by the face image acquisition process S3.

The blood pressure stage determination process S43 is a process of determining the blood pressure stage of the subject P based on the spatial feature amount extracted by the spatial feature amount extraction process S42 and the determination spatial feature amount.

The estimated blood pressure stage output process S44 is a functional block that outputs the determination result obtained by the blood pressure stage determination process S43 as the estimation result ES of the blood pressure stage of the subject P.

As shown in FIG. 10, the learning process S5 includes a learning data storage process S51, a feature amount extraction process S52, and a feature amount learning process S53.

The learning data storage process S51 is a process of storing a plurality of learning face images labeled corresponding to the blood pressure stages consisting of two or three stages.

The feature amount extraction process S52 is a process of extracting the spatial feature amount of the learning face image using the learned model TM.

The feature amount learning process S53 is a process of changing network parameters of the learned model TM based on a relationship between the extraction result obtained by the feature amount extracting process S52 and a label attached to the learning face image serving as the extraction target thereof such that extraction accuracy of the spatial feature amount by the feature amount extraction process S52 becomes high.

### [Operation/Advantages]

In the blood pressure estimation system 200 according to the second embodiment configured as described above, the face image FI of the subject P is captured by the face image acquisition device 210. The captured face image FI is input to the blood pressure estimation device 220. The blood pressure estimation device 220 extracts the spatial feature amount of the input face image FI, determines the blood pressure stage of the subject P based on the extracted spatial feature amount and the determination spatial feature amount, and outputs the determination result as the estimation result ES of the blood pressure stage of the subject P.

According to the blood pressure estimation system 200, the blood pressure stage of the subject P can be instantaneously estimated in a non-contact manner based on the spatial feature amount of the face image FI of the subject P. More specifically, when one face image FI of the subject P is only captured, the blood pressure of the subject P can be instantaneously and accurately estimated.

Furthermore, the blood pressure estimation system 200 according to the second embodiment uses the spatial feature amount extracted by the learning device 230 as the determination spatial feature amount. The learning device 230 stores a plurality of learning face images labeled corresponding to the blood pressure stages consisting two or three blood stages, extracts the spatial feature amount of the face image FI of the subject from the learning face image by using the learned model TM, and changes the network parameters of the learned model TM based on a relationship between the extraction result and a label attached to the learning face image serving as the extraction target thereof such that extraction accuracy of the spatial feature amount of the face image FI of the subject P becomes high. When the learning of the learned model TM progresses, the extraction accuracy of the spatial feature amount of the face image FI is improved, and the accuracy of the spatial feature amount stored in the determination feature amount storage unit 121 is also improved.

Therefore, according to the blood pressure estimation system 200, when the learning of the learned model TM in the learning device 230 progresses, the estimation accuracy of the blood pressure stage can be improved.

The present invention is not limited to the above embodiments. For example, although the blood pressure estimation system 200 includes the learning device 230 in the second embodiment, the learning device 230 can be omitted. When the learning device 230 is omitted, a spatial feature amount extracted or generated by means other than the learning device 230 is stored in the determination feature amount storage unit 221 of the blood pressure estimation device 220.

### EMBODIMENTS

### Example of the First Embodiment

### 1. Purpose of Experiment

In Japan, increasing number of patients with lifestyle-related diseases has become a social problem. Lifestyle-related diseases are diseases caused by lifestyle habits such as irregular eating habits, overeating, and lack of exercise, and it is necessary to understand their own health condition on a daily basis. As a countermeasure, there is a measure to monitor vital signs such as blood pressure on a daily life. The vital sign means a “proof of living”, and mainly shows four indicators of a blood pressure, a heart rate, respiration, and a body temperature.

Vital signs are often used as indicators of health status, and daily monitoring of vital signs is expected to lead to early detection of diseases such as lifestyle-related diseases. In order to realize daily vital sign monitoring, unconstrained, unconscious, and non-contact measurement is essential, and many studies have been conducted. In previous studies of non-contact blood pressure measurement, a blood pressure estimation model was constructed by performing a regression analysis between a nasal skin temperature and a blood pressure acquired by an infrared thermography camera.

However, it has been understood that not only local regions such as the skin temperature of the nose but also a large facial regions are associated with psychological/physiological conditions. On the other hand, a method of clarifying a causal relationship between independent components and physiological/psychological indexes by extracting a feature amount by applying an independent component analysis (independent component image: to be referred to as ICA hereinafter) to a face thermal image (facial thermal image: to be referred to as FTI hereinafter) has been proposed. Therefore, the purpose of this study is to develop a blood pressure estimation technique based on the independent components of the face thermal image, and multiple regression analysis of the independent components extracted by applying ICA to FTI and the mean blood pressure (Mean blood pressure: to be referred to as MBP hereinafter) is performed to try extraction of a feature amount related to a blood pressure and blood pressure estimation.

### 2. Experiment Method

### <2.1> Experiment Environment

A subject was one healthy adult male (22 years old), and the measurement was carried out in a convection-free shielded room at 24.5° C. Experiment contents, purpose, and target of the survey were sufficiently explained to the subject in advance to the subject by parol and in writing, and the agreement for the experiment cooperation was confirmed by signing. The subject was entered in the shield room 15 minutes before the measurement and acclimatized to the room temperature. As the measurement items, MBP, cardiac output (CO), total peripheral vascular resistance (PR), and heart rate (HR) were measured by using a FTI continuous blood pressure monitor. The FTI was measured by placing an infrared thermography device (TVS 200EX; available from AVIONICS Co., Ltd.) at a position 1.2 m in front of the subject. Thermal image size was 320×240 pixels, and a temperature resolution was 0.1° C. The facial skin emissivity was set to ε=0.98, and the thermal image was recorded at a sampling frequency of 1 Hz. As the continuous blood pressure monitor, a finger cuff of a continuous blood pressure/hemodynamic instrument (Finometer model 2, Finapress MedicalSystems; available from BV Corporation) was attached to the second joint of the middle finger of the left hand of the subject, and the continuous blood pressure was measured and recorded on a PC at a sampling frequency of 1 Hz.

### <2.2> Measuring Method

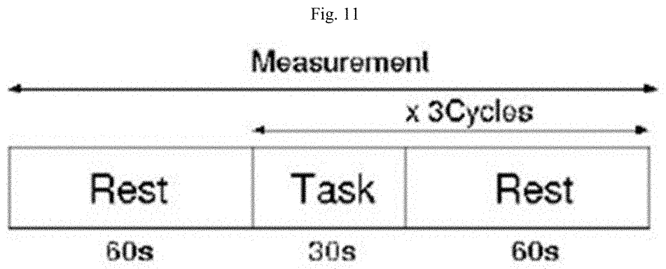

The experimental protocol is shown in FIG. 11. The experiment consists of rest (Rest) for 60 seconds and Valsalva test (Valsalva maneuve: to be referred to as Valsalva hereinafter) for 30 seconds. The Valsalva (Task) was performed 3 times, and Rests was provided before and after that. The experimental time was 330 seconds. The experiment was performed in an always closed-eye state. The FTI continuous blood pressure monitor measured from the start of the experiment to the end of the experiment.

### 3. Analyzing Method

<3.1> An FTI region was extracted from the thermal image recorded by ICA with a×b pixels so that the hair and the background were not reflected as shown in FIG. 9. Then, the FTI for 330 seconds extracted in one experiment is developed into a one-dimensional FTI vector x(t)=[x₁, x_{z}, . . . , xₖ] (t=1, 2, . . . , 330) (k=1, 2, . . . , a×b), and an FTI vector for 330 seconds is similarly stored in the following matrix (1).

$$\begin{matrix}[ Numerical Expression 1 ] & \\ X = [ \begin{matrix}{ϰ}_{1 , 1} & \text{...} & {ϰ}_{1 , a \times b} \\ ⋮ & ⋱ & ⋮ \\ {ϰ}_{330 , 1} & \text{...} & {ϰ}_{330 , a \times b}\end{matrix} ] & ( 1 )\end{matrix}$$

The resultant value was used as an observation signal x, and the ICA was applied. The ICA

[Numerical Expression 2]

X=AS (2)

In the relational expression (2) described above, a mixing matrix A estimating an independent component matrix S is calculated from the observation signal x. When the ICA is applied to the observation signal x, the independent component matrix S and the mixing matrix A are estimated as follows:

$$\begin{matrix}[ Numerical Expression 3 ] & \\ S = {[ {s}_{1} , {s}_{2} , ⋯ , {s}_{k} ]}^{T} = [ \begin{matrix}{s}_{1 , 1} & \text{...} & {s}_{1 , n} \\ ⋮ & ⋱ & ⋮ \\ {s}_{a \times b , 1} & \text{...} & {s}_{a \times b , t}\end{matrix} ] & ( 3 ) \\ [ Numerical Expression 4 ] & \\ A = [ {a}_{1} ( t ) , {a}_{2} ( t ) , ⋯ , {a}_{n} ( t ) ] = [ \begin{matrix}a & \text{...} & {a}_{1 , n} \\ ⋮ & ⋱ & ⋮ \\ {a}_{330 , 1} & \text{...} & {a}_{330 , t}\end{matrix} ] & ( 4 )\end{matrix}$$

The n was the number of independent components. As described above, in the independent component matrix S, the feature amount of the facial skin temperature is estimated as a matrix of the feature amount (a×b)×n of the facial skin temperature, and the mixing matrix A is estimated as the matrix of the contribution n×t of each independent component sn to the observation signal x. In the following, a component an(t) of the mixing matrix corresponding to each component is called a weighted time series.

In the ICA, it is assumed that the number of observation components is equal to the number of independent components. In fact, the number of independent components is usually unknown, and there is often no clear number of independent components. Here, the number of independent components is set to 14.

### <3.2> Multiple Regression Analysis

The causal relationship with the independent components has been cleared such that the weighted time series obtained by ICA is used as an explanatory variables and MBP is used as the objective variable. In the variable selection of the multiple regression analysis, after the model was optimized by the stepwise method, the model was optimized until the P value of the explanatory variable was 0.05 or less. In order to confirm the presence/absence of multicollinearity, when there was a component with a dispersion expansion coefficient (VIF) of 5.0 or more, the component was removed. After removing the component, the explanatory variables were optimized again.

### 4. Experiment Result and Discussion

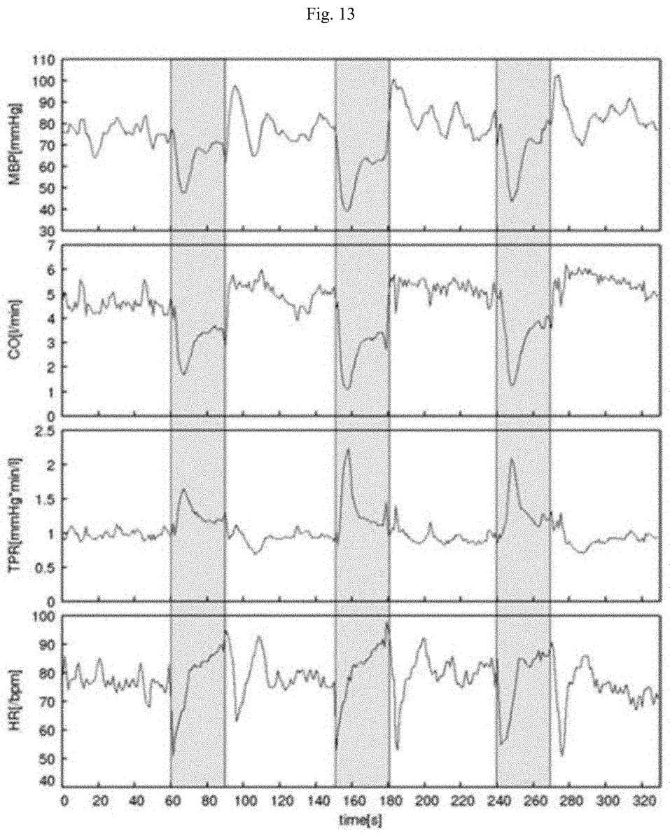

FIG. 13 shows time series changes from the start to the end of the experiment of the MBP, CO, TPR, and HR. The hatched area indicates the section during the Valsalva test. As a result, immediately after the start of the Valsalva test, HR decreased and the amount of blood pumped from the heart also decreased. Therefore, CO decreased, MBP changed, and TPR increased due to sympathetic nervous system hyperactivity. After the Valsalva test was completed, CO and TPR returned to those in the state before the test, and MBP increased. From these results, it was shown that the mechanism of blood pressure change during the Valsalva test is different from that after the Valsalva test.

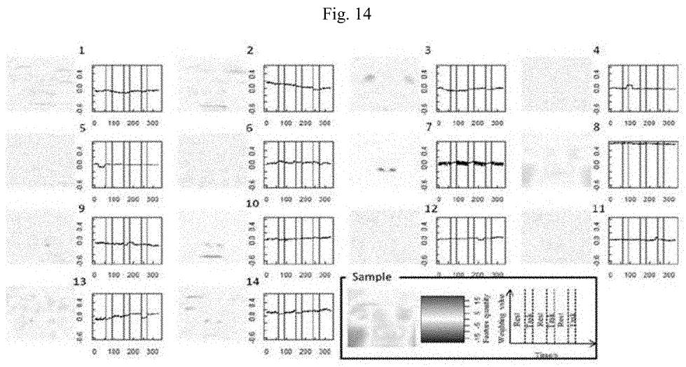

FIG. 14 shows the independent components obtained by applying ICA to the observation signals of FTI from the start to the end of the experiment, and the weighted time series corresponding thereto. As a result, feature amounts appeared on various regions of the face such as the eighth component on both cheeks and the tenth component on the lips. On the other hand, in the seventh component, the feature amount appears in the nostril, and the weighted time series of the component repeats a rapid change in the Rest section. From this result, the seventh component was considered as the respiratory component produced by the Valsalva test, and the component was removed from the explanatory variables of the multiple regression analysis. Therefore, multiple regression analysis was performed using the mean blood pressure and the weighted time series extracted by ICA.

Table 1 shows partial regression coefficients obtained by the multiple regression analysis of MBP and the weighted time series. As a result, a correlation (R²=0.58) was recognized between the MBP and the weighted time series. Therefore, a standard partial regression coefficient was calculated in order to confirm an independent component having a high contribution rate to the objective variable MBP. The results are shown in Table 2. The standard partial regression coefficients of the 13th and 14th components were higher than those. As a result, it was suggested that the feature amount that appeared on the whole face and around the right eye and the right eyebrow were related to the blood pressure.

TABLE 1

| | The partial regression coefficients Bn corresponding to the weighted time series An and the constant term | | | | | | | | | | | | | | | |
|---|---|---|---|---|---|---|---|---|---|---|---|---|---|---|---|---|
| | B₁ | B₂ | B₃ | B₄ | B₅ | B₆ | B₇ | B₈ | B₉ | B₁₀ | B₁₁ | B₁₂ | B₁₃ | B₁₄ | Cast | R² |
| MBP | 142.95 | 64.71 | −93.63 | 59.85 | — | −100.10 | — | −173.3 | 150.8 | — | −86.27 | −71.18 | 78.12 | 211.28 | 182.13 | 0.58 |

TABLE 2

| | The standard partial regression coefficients Cₙ corresponding to the weighted time series Aₙ and the constant term | | | | | | | | | | | | | |
|---|---|---|---|---|---|---|---|---|---|---|---|---|---|---|
| | C₁ | C₂ | C₃ | C₄ | C₅ | C₆ | C₇ | C₈ | C₉ | C₁₀ | C₁₁ | C₁₂ | C₁₃ | C₁₄ |
| MBP | 0.29 | 0.34 | −0.28 | 0.15 | — | −0.18 | — | −0.17 | 0.34 | — | −0.19 | −0.12 | 0.42 | 0.67 |

The standard partial regression coefficients corresponding to the weighted time series and the constant term

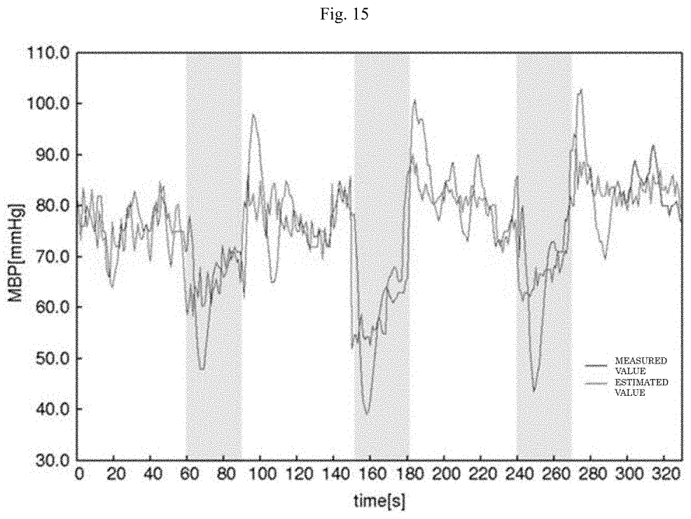

FIG. 15 shows the results of MBP estimated values obtained by multiple regression analysis and the measured values. The result of calculating the mean square error between the estimated value and the measured value was 54.99 mmHg. A large error was found between the estimated value and the measured value when the MBP sharply changed immediately after the start of the Valsalva test and immediately after the end thereof. It is conceivable that the accuracy of blood pressure estimation will be improved by extracting a feature amount related to a sharp change in MBP.

### 5. Conclusion

In this experiment, aiming at the development of blood pressure estimation technique based on the independent components of the face thermal image, multiple regression analysis of the independent component extracted by applying ICA to FTI and MBP is performed to try to extract the feature amount related to blood pressure and to estimate the blood pressure. As a result, a correlation (R²=0.58) was observed between the weighted time series extracted by ICA and the MBP, it was suggested that the feature amounts appearing on the entire face and near the right eye and right eyebrow were related to the blood pressure.

### Example of the Second Embodiment

### 1. Purpose of Experiment

The purpose is the same as that of the first embodiment.

### 2. Experiment Method

In the experiment, a cold pressurization test (cold pressure test: to be referred to as CPT hereinafter) for promoting different blood pressure change mechanisms and a Valsalva test were carried out. The subjects were 7 healthy adults (4 males, 3 females, 21.4±0.5 years old) for CPT, and 7 healthy adults (4 males, 3 females, 1.6±0.5 years old) for Valsalva. As the measurement index, FTI was measured with an infrared thermography camera (TVS-200EX; available from AVIONICS corporation), and MBP was measured with a continuous blood pressure monitor (Finometer model 2, Finapress Medical Systems available from By). The experiment was performed in the state in which the subjects sat in chairs and with always closed eyes. In CPT, the right hands of the subjects were put in water at 12° C., and in Valsalva, the subjects held their breathes. The experiment consisted of resting closed eyes for 60 seconds and blood pressure change test for 30 seconds, and was repeated 3 times.

In addition, a performance evaluation experiment was performed to evaluate the performance of a derived general model. The test subjects were 5 healthy adults (4 males, 1 female, 23.0±3.5 years old) who did not participate in the Valsalva and CPT experiments in an experimental laboratory. The experiment consisted of only resting closed eyes for 30 seconds, and FTI and MBP were measured.

### 3. Analyzing Method

### <3.1> Individual Model Derivation

An FTI region was extracted with a×b pixels from the measured thermal image so that the hair and background were not reflected. Each extracted FTI was subjected to mosaic processing with 2×2 pixels, and then a low-pass filter of 0.1 Hz was applied to remove body motion components. Then, the FTI was developed into a one-dimensional vector, and the FTI vector for t seconds of experiment was stored in the matrix X shown in the above-mentioned expression (1).

In this embodiment, independent component analysis was performed as a method of extracting independent signals from mixed signals. The independent component analysis can be expressed by the above expression (2), where X is an observation signal, A is a contribution to the independent component, and S is an independent component.

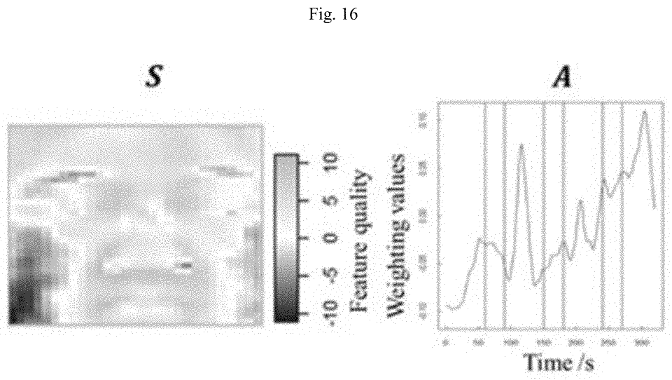

In this embodiment, X was applied to ICA as an observation signal. An example of the application result is shown in FIG. 16. S is estimated as a matrix of FTI feature amounts, and A is estimated as a matrix of an observation signal of each independent component and contribution to X. Hereinafter, the independent component A will be called a weighted time series. A moving mean filter for 10 seconds was applied to the time series data of MBP and A to perform smoothing. The facial skin temperature changes depending on the speed and force of the skin blood flow. Therefore, in order to consider the speed and force of blood flow, a weighted time series first-order differential value A′ and a weighted time series second-order differential value A″ are calculated, and explanatory variables MBP objective variables were given by A, A′, and A″ to derive a blood pressure estimation individual model.

### <3.2> Derivation of General Model

Independent components related to the blood pressure were extracted from the derived blood pressure estimation individual model and aggregated in a matrix. The matrix is defined as S_{BP}.

$${S}_{BP} = [ \begin{matrix}{S}_{1 , 1} & \text{...} & {S}_{1 , c \times d} \\ ⋮ & ⋱ & ⋮ \\ {S}_{n , 1} & \text{...} & {S}_{t , c \times d}\end{matrix} ] \qquad [ Numerical Expression 5 ]$$

The S_{BP} unified the number of pixels into c×d pixels, and then normalized each image so that the maximum value was 1 and the minimum value was 0. In order to remove similar independent components from the aggregated matrix, a correlation analysis is applied to each image to remove the image when R>0.6, and the weighted time series Ae shown in the following expression (6) was calculated.

[Numerical Expression 6]

Ae=XS⁻¹ (6)

Ae′ and Ae″ were calculated from the weighted time series Ae, a blood pressure estimation model was derived using the explanatory variables as MBPs of all the subjects, and the objective variables as Ae, Ae′, and Ae″, and the model is defined as a general model. In this study, CPT, Valsalva, and independent components related to blood pressure extracted from hybrids of the CPT and the Valsalva were aggregated to construct three types of general models, and the three models were compared with each other.

### 4. Result and Discussion

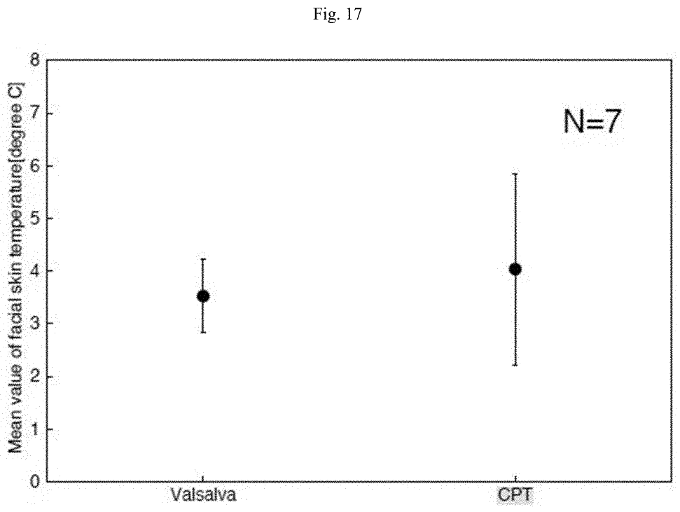

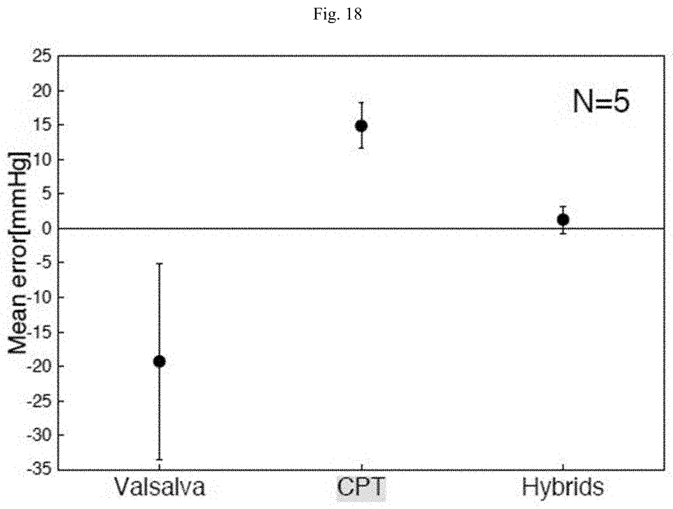

FIG. 17 shows the derivation result of the blood pressure estimation individual model estimated from the FTIs of Valsalva and CPT. The abscissa indicates each Task, and the ordinate indicates the mean square error. From FIG. 17, it has been clarified that a blood pressure is estimated with almost the same accuracy in Valsalva and CPT, and that MBPs of both blood pressure change mechanisms can be estimated by this method. The general models of Valsalva, CPT, and Hybrids were derived using the independent components extracted by the individual model. The performance of the general model was evaluated using unknown data. The result is shown in FIG. 18. The abscissa indicates the type of general model, the ordinate indicates the mean error, and the baseline indicates that the error is zero. From FIG. 18, blood pressures were estimated to be low for Valsalva and high for CPT, and estimated with the highest accuracy for Hybrids with a mean error of about 1.26 mmHg. From these results, it was clarified that the blood pressure can be estimated by aggregating the independent components related to the two blood pressure change mechanisms and deriving a general model.

### 5. Conclusion

In this embodiment, for the purpose of blood pressure estimation based on the facial skin temperature independent component, a blood pressure estimation individual model was derived using the independent components extracted by applying ICA to FTI, and the blood pressure estimation general model was derived by aggregating the independent component related to the blood pressure extracted from the individual model. As a result, the individual model could be derived using the method regardless of the blood pressure change mechanisms, and the accuracy of the general model constructed from independent components aggregated from the individual models of both the blood pressure change mechanisms was highest, i.e., 1.26 mmHg. In this manner, it became clear that the blood pressure can be estimated by using the method of the embodiment.

### Example of the Third Embodiment

### 1. Purpose of the Embodiment

Hypertension, which is one of the lifestyle-related diseases, is a risk factor for cerebrovascular and cardiovascular disorders among the three major causes of death in Japan. According to the patient survey of Ministry of Health, Labor and Welfare in japan in 2014, the estimated total number of patients with hypertensive cardiovascular diseases has tended to increase since 2008. In the modern age, which is a super-aged society, the deterioration of life functions of the elderly due to the aggravation of cardiovascular diseases of the elderly has become a problem. Early detection of lifestyle-related diseases including hypertension and preventing measures against aggravation of the diseases have been promoted.

According to the guidelines of the Japanese Society of Hypertension, blood pressures are classified into a normal region blood pressure and a hypertension on the basis of a measured systolic blood pressures and a measured diastolic blood pressure, and hypertension is subdivided into three stages of I-degree hypertension, II-degree hypertension and III-degree hypertension. The above blood pressure stages are widely used as diagnostic criteria for hypertension.

In addition, a clinic blood pressure measurement is used for the diagnostic criteria of hypertension and to determine severity of hypertension. However, the presence of white coat hypertension where a blood pressure rises due to temporary tension at medical institutions, masked hypertension where a normal blood pressure is exhibited at medical institutions but hypertension is exhibited at home poses problems.

Continuous monitoring of vital signs is essential in daily life, including at home, to detect hypertension early and to prevent aggravation thereof. In order to realize the continuous monitoring of vital signs in daily life, a technique of unconsciously and low-invasively sensing vital signs is needed. In recent years, low-invasive vital sign sensing technique such as a technique of estimating the heart rate of the subject from a pulse wave of the fingertip plethysmogram captured by the camera of a smart phone or a pulse wave component extracted from a face visible image has been established.

In order to establish a non-contact blood pressure sensing technique, we tried to perform correlative analysis between a nasal skin temperature measured in a non-contact manner and a mean blood pressure and correlative analysis between the amplitude/phase of the pulse wave component of the nose region extracted from a face visible image and the mean blood pressure. In a conventional research, an analysis target area was limited to the nose area, but by extending the analysis target area to the entire face area and extracting features related to blood pressure from the entire face area, improvement in blood pressure estimation accuracy can be expected. Furthermore, in recent years, studies have been conducted to apply deep learning algorithms to biometric information, and an attempt to construct feature extraction and drowsiness stage estimation model related to drowsiness stages is executed by applying the deep learning algorithms to a facial skin temperature distribution.

If the features related to a blood pressure can be extracted and a blood pressure stage can be estimated by applying the deep learning algorithms to the facial skin temperature distribution when blood pressure changes, it can be expected to lead to the realization of non-contact diagnosis of hypertension and continuous monitoring of vital signs in daily life.

In this example, aiming at the realization of continuous monitoring of vital signs using a non-contact sensing technique, by applying deep learning algorithms to a facial skin temperature distribution measured in a state where blood pressure is changed, an attempt to construct an individual model which extracts features related to blood pressure and estimates a blood pressure stage based on the features was executed.

### 2. Blood Pressure Change Examination by Cold Load Test

A cold load test was conducted as a blood pressure change experiment. During the cold load test, the mean blood pressure was measured using a continuous sphygmomanometer simultaneously with the measurement of a facial skin temperature distribution using an infrared thermography camera.

### <2.1> Experiment Conditions

Subjects were 6 healthy adult males and 1 female (age: 22.2±2.9 years). The experiment was performed in a laboratory in an office lighting environment of 900 to 1000 lux illuminance at a room temperature of 25±1° C. The experiment was performed during the day in consideration of an influence of circadian rhythm.

### <2.2> Experimental Environment

The experimental environment consists of a chair for the subject to sit on, a table, a constant temperature water tank for cold load test (NCB-2510, TOKYO RIKAKIKAI CO, LTD), and an infrared thermography camera (TV-200EX, NIPPON AVIONICS CO., LTD.). The table was placed in front of the subject, and the constant temperature water tank was placed to the right of the table. Since the target region of the cold load test was set to the right hand of the subject, the constant temperature water tank was arranged so that the right wrist of the subject was immersed in the water in the water tank. A towel for wiping the right hand of the subject was placed on the table. The infrared thermography camera was placed at a location 70 cm away from the face of the subject so that the facial skin temperature distribution of the subject could be measured.

### <2.3> Experiment Method

In order to adjust the facial skin temperatures of the subjects to the room temperature of the laboratory, the subjects were allowed to enter the laboratory at least 15 minutes before the start of the experiment. After entering the laboratory, the subjects were given a full explanation of the experiment outline and consent was obtained to participate in the experiment. After obtaining the consent to participate in the experiment, a blood pressure measuring cuff of a continuous blood pressure monitor (Finometer model 2, Finapress Medical Systems) was attached to the intermediate region between the first and second joints of the left middle finger of the subject. In the experiment, one set consisted of a resting closed eye for 2 minutes and a cold load test for 1 minute, and a total of 3 sets were performed, and then a resting closed eye was performed for 1 minute to finish the experiment. During the experiment, the subject was kept in a sitting position. In the cold load test, the subject is instructed to put her/his right wrist in the water in the constant temperature tank kept at a temperature of 14° C. so that her/his wrist is immersed in the water. After finishing the cold load test, the subject was instructed to put her/his right hand on the towel on the table.

The facial skin temperature distribution was measured at 1 fps. The size of the face thermal image containing the facial skin temperature information was 240 pixels vertically and 320 pixels horizontally, and the thermal emissivity of the skin was 0.98. On the other hand, the sampling frequency of the continuous blood pressure monitor was set to 1 Hz. With respect to the time series data of the average blood pressure measured by the continuous blood pressure monitor, in order to reduce the effect of a sudden change in a measured value of the continuous blood pressure monitor due to the body movement of the subject, data obtained by applying a moving average every 20 seconds to the measured value output from the continuous blood pressure monitor was used as analysis data of the mean blood pressure.

### 3. Blood Pressure Stage Estimation by Deep Learning

In this example, construction of a personal model for blood pressure stage estimation by applying a deep learning algorithm using a convolutional neural network (Convolutional Neural Network: CNN) to a facial skin temperature distribution measured in a blood pressure change experiment was tried. CNN was constructed using MATLAB (registered trademark) 2018a (available from The MathWorks, Inc).

### <3.1> Definition of Blood Pressure Stage

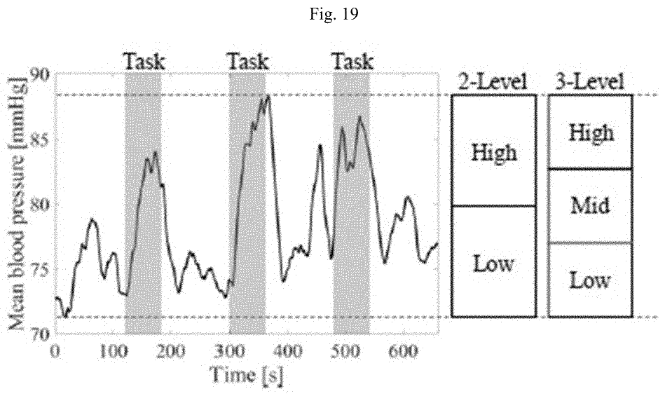

Here, FIG. 19 shows a representative example of time series of a blood pressure change. However, the abscissa indicates the elapsed time after the start of the first rest closed eye, and the ordinate indicates a mean blood pressure after moving average every 20 seconds. Task in the figure is a cold load test section, and it will be clear that the mean blood pressure in the cold load test section is higher than the mean blood pressure in the rest-closed eye section. The minimum value of the mean blood pressure of a subject G shown in FIG. 19 was 71.3 mmHg, and the maximum value was 88.4 mmHg. Table 3 shows the maximum and minimum values of the mean blood pressure in the experiments of all the subjects.

TABLE 3

| Sub. | A | B | C | D | E | F | G |
|---|---|---|---|---|---|---|---|
| Max | 117.8 | 120.3 | 93.9 | 107.5 | 111.8 | 98.9 | 88.4 |
| Min | 95.8 | 87.4 | 74.7 | 80.9 | 92.8 | 80.8 | 71.3 |

In the present example, two stages of dividing the average blood pressure value during the experiment into Low level/High level and three stages of dividing into Low level/Middle level/High level were defined as blood pressure stages. In the two stages, the mean blood pressure during the experiment was set as Low level when it was lower than an intermediate value between the minimum and maximum values of the mean blood pressure, and as High level when it was higher than the intermediate value. On the other hand, in the three stages, the change range of the mean blood pressure during the experiment was divided into three equal parts, the low range of the three parts was defined as a low level, the intermediate rage was defined as a middle range, and the high range was defined as High level.

### <3.2> Input Data Creation of Deep Learning

A facial skin temperature distribution measured during the blood pressure change experiment was used as input data for CNN.

A binarized image with a constant temperature value as a threshold value was created from a face thermal image containing information of the facial skin temperature distribution. After a face area was specified from the binarized image, the image is trimmed to 200 pixels vertically and 200 pixels horizontally to create an image containing only the information of the facial skin temperature distribution. Skin temperature distributions were classified into blood pressure stages based on the mean blood pressure value measured simultaneously. Data is expanded by randomly rotating the facial skin temperature distribution at each blood pressure stage by −15 to 15° about the center of the facial skin temperature distribution, and then thinned out to make the numbers of input data at stages equal to each other.

<3.3> In CNN configuration and optimal deep learning of hyperparameters, hyperparameters such as the number of layers of CNN and a filter size may significantly affect the estimation accuracy, but it is difficult to select and control the optimal hyperparameters. In recent years, methods such as a Grid search method and a Randomized search method that automatically control the hyperparameters have been used. Here, the hyperparameters are optimized by Bayesian optimization.

The CNN consisted of an input layer, a convolutional layer, a normalization layer, a pooling layer, a full connecting layer and an output layer. The filter size of each convolution layer was set to 3, the stride was set to 1, and the padding size was set to 1, and the number of filters of the convolution layer in the nth layer was set to 32×2ⁿ¹. On the other hand, the size of each pooling layer was 2, the stride was 2, and the pooling method was maximum value pooling. After batch normalization between each convolutional layer and each pooling layer is performed, normalized linear function was applied. The number of elements in the full connecting layer was set to 2 and 3 corresponding to the blood pressure stages, and an activation function was a softmax function.

The Bayesian optimization was applied to CNN, which is an individual model of each subject. The hyperparameters optimized by Bayesian optimization were the number of convolutional layers, an initial learning rate, a momentum, and an L2 normalized strength. As search ranges of the hyperparameter, the number of convolutional layers was 3 to 15, the initial learning rate was 0.0001 to 0.05, the momentum is 0.8 to 0.95, and the L2 normalized strength was 10⁻¹⁰ to 0.01.

In Bayesian optimization, the facial skin temperature distribution of each of the subjects was used as input data, 80% of all input data were training data, and the remaining 20% were test data. The above-mentioned input data was learned by the CNN with each hyperparameter, and the classification error serving as the output was used as the objective function of Bayesian optimization. The optimal hyperparameters were determined by minimizing the objective function. As a result of the Bayesian optimization, the optimal CNN configurations for the subjects were the same (see Table 4).

TABLE 4

| | Activation | | |
|---|---|---|---|
| Layers | function | Size | Stride |
| Conv1 | ReLU | 3 × 3 | 1 |
| BatchNorm1 | — | — | — |
| MaxPooling1 | — | 2 × 2 | 2 |
| Conv2 | ReLU | 3 × 3 | 1 |
| BatchNorm2 | — | — | — |
| MaxPooling2 | — | 2 × 2 | 2 |
| Conv3 | ReLu | 3 × 3 | 1 |
| BatchNorm3 | — | — | — |
| MaxPooling3 | — | 2 × 2 | 2 |
| FC | Softmax | N-Class | — |

In Table 4, Cony is a convolutional layer, BatchNorm is a batch normalization layer, MaxPooling is a maximizing pooling layer, FC is a full connecting layer, ReLU is a normalized linear function, and Softmax is a softmax function. The size of the full connecting layer is 2 in the two stages and 3 in the three stages. Furthermore, optimum hyperparameters for each of the subjects are shown in Tables 5 and 6. Hyperparameters showed different values for the subjects, respectively.

TABLE 5

| Subject | Learn rate | Momentum | L2 regularization |
|---|---|---|---|
| A | 1.07 × 10⁻⁵ | 0.801 | 4.17 × 10⁻³ |
| B | 2.65 × 10⁻⁴ | 0.908 | 9.09 × 10⁻³ |
| C | 1.00 × 10⁻⁴ | 0.837 | 3.86 × 10⁻⁴ |
| D | 1.08 × 10⁻⁴ | 0.841 | 2.87 × 10⁻⁴ |
| E | 1.02 × 10⁻⁵ | 0.900 | 1.06 × 10⁻¹⁰ |
| F | 1.20 × 10⁻⁴ | 0.896 | 2.46 × 10⁻⁷ |
| G | 4.36 × 10⁻⁴ | 0.801 | 1.22 × 10⁻⁹ |

TABLE 6

| Subject | Learn rate | Momentum | L2 regularization |
|---|---|---|---|
| A | 2.75 × 10⁻⁴ | 0.830 | 1.56 × 10⁻⁹ |
| B | 2.30 × 10⁻³ | 0.802 | 5.30 × 10⁻³ |
| C | 4.11 × 10⁻⁴ | 0.826 | 8.88 × 10⁻⁸ |
| D | 4.47 × 10⁻³ | 0.759 | 2.88 × 10⁻⁸ |
| E | 2.87 × 10⁻³ | 0.770 | 1.16 × 10⁻⁸ |
| F | 1.14 × 10⁻³ | 0.797 | 4.94 × 10⁻⁵ |
| G | 1.49 × 10⁻³ | 0.794 | 4.94 × 10⁻⁵ |

### <3.4> Deep Learning of Facial Skin Temperature Distribution

A personal model for blood pressure stage estimation was constructed by using the facial skin temperature distribution of each subject as input data and applying the CNN optimized by Bayesian optimization.

A learning rule of CNN is an error back propagation method. The learning was completed when the error between an output at the time of inputting the training data and the objective variable, which is the blood pressure stage, became 20%. During learning, 80% of all the input data was used as training data, and the remaining 20% was used as test data, and cross-validation was performed 5 times. Accuracy verification was performed 10 times for each cross-verification, and a final correct answer rate was obtained by averaging the correct answer rates obtained in each cross-verification.

### 4. Results and Discussion

### <4.1> Change of Mean Blood Pressure by Blood Pressure Change Experiment

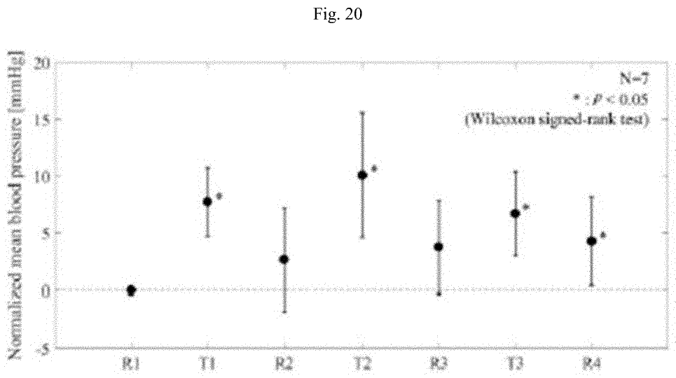

FIG. 20 shows the mean blood pressure displacement in the blood pressure change experiment. The abscissa indicates a resting closed eye (R) section and a cold load test (T) section in the experiment, and the ordinate indicates the mean blood pressure displacement when a first resting closing eye (R1) section is the baseline. The plot in FIG. 20 shows the mean value of the subject, and the error bar shows a standard deviation. Furthermore, as a result of the Wilcoxon sign rank test, it was cleared that the mean blood pressure displacement in all the cold load tests (T1, T2, and T3) section and a fourth resting closed eye (R4) section was significantly higher than the baseline. (*: p<0.05). The results of conventional researches have shown that total peripheral vascular resistances increase in the cold load test, and it is considered that the increase in mean blood pressure in the cold load test is due to the increase of the total peripheral vascular resistances.

### <4.2> Correct Answer Rate of Blood Pressure Stage Estimation

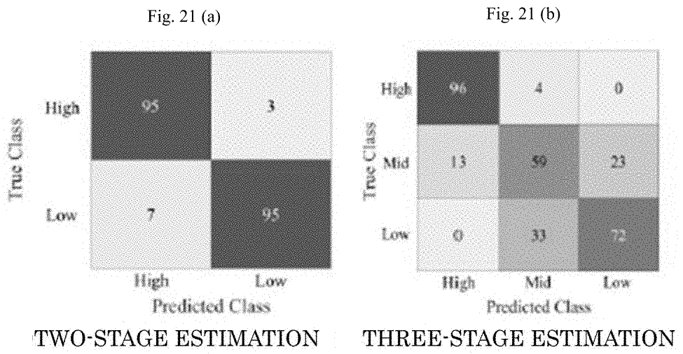

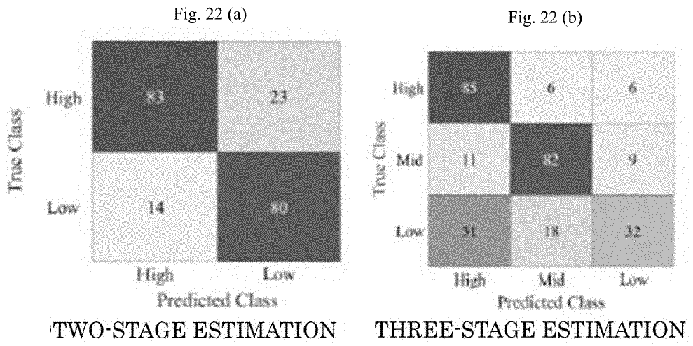

FIGS. 21(A) and 21(B) and FIGS. 22(A) and 22(B) show representative examples of the confusion matrix in the blood pressure stage estimation in two stages and three stages. The vertical of the confusion matrix is the actual stage, and the horizontal of the matrix is the estimated stage. FIGS. 21(A) and 21(B) show the confusion matrix of a subject A having the highest correct answer rate in the two-stage estimation, and FIGS. 22(A) and 22(B) show the confusion matrix of a subject G having the lowest correct answer rate in the two-stage estimation. In the two-stage estimation of the subject A, a result in that estimation can be performed at a high level is shown. On the other hand, in the three-stage estimation, a result in that classification between the Middle level and the Low level is particularly difficult is shown. On the other hand, in the two-stage estimation of the subject G, a result in that samples estimating the skin temperature distribution at the High level as a Low level are many is obtained, and in the three-stage estimation, a result in that samples estimating the skin temperature distribution at the Low level as a High level are many is obtained. Table 7 shows correct answer rates of blood pressure stage estimation for all subjects. Regarding the correct answer rates of all the subjects, the correct answer rate is 80% or more in the two-stage estimation, while the correct answer rate is about 65 to 85% in the three-stage estimation, which is lower than that in the two-stage estimation.

TABLE 7

| Subject | 2-Level | 3-Level |
|---|---|---|
| A | 95.5 | 75.7 |
| B | 94.0 | 85.7 |
| C | 82.0 | 80.7 |
| D | 89.0 | 82.6 |
| E | 94.5 | 77.3 |
| F | 87.0 | 73.7 |
| G | 81.5 | 66.3 |

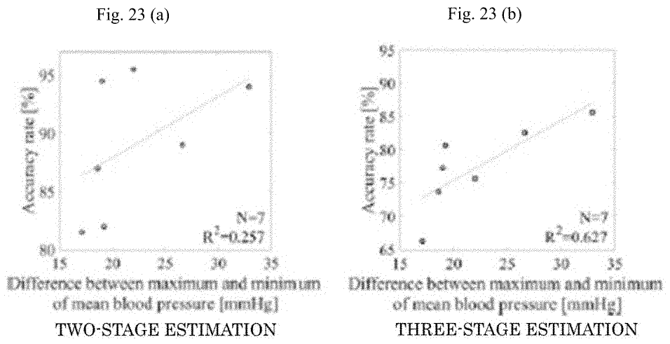

Here, FIGS. 23(A) and 23(B) show a scatter diagram and a regression line of the difference between the maximum and minimum values of the mean blood pressure in the blood pressure change experiment and the correct answer rate of the blood pressure stage estimation. The abscissa indicates the difference between the maximum and minimum values of the mean blood pressure, and the ordinate indicates the correct answer rate for blood pressure stage estimation. Further, the straight line in the figure indicates a linear regression line, and R2 indicates a regression coefficient. In both the results of 2-stage estimation and 3-stage estimation, when the difference between the maximum and minimum values of the mean blood pressure is small, the correct answer rate of the blood pressure stage estimation tends to decrease. In particular, a moderate correlation was observed between the difference between the maximum and minimum value of the mean blood pressure and the correct answer rate of the 3-stage estimation. The subject G, who had the lowest correct answer rate in the 2-stage estimation and the 3-stage estimation, had a difference between the highest and lowest mean blood pressures of 17.1 mmHg, which was the lowest value among all the subjects. When the blood pressure stage is defined, the difference between the maximum and minimum values of the mean blood pressure is divided into two equal parts or three parts. Thus, the smaller the difference between the maximum value and the minimum value of the mean blood pressure, the smaller the difference between the blood pressure values in the respective stages. It is considered that, since the smaller the blood pressure change is, the smaller the facial skin temperature distribution change is, the correct answer rate of the blood pressure stage estimation of the subject G having the lowest difference between the highest and lowest mean blood pressures becomes low.

### <4.3> Feature Map Analysis in CNN

In this example, the feature map in the convolutional layer filter of CNN was analyzed to extract the features related to blood pressure on the face, and the feature maps at blood pressure stages were compared with each other.

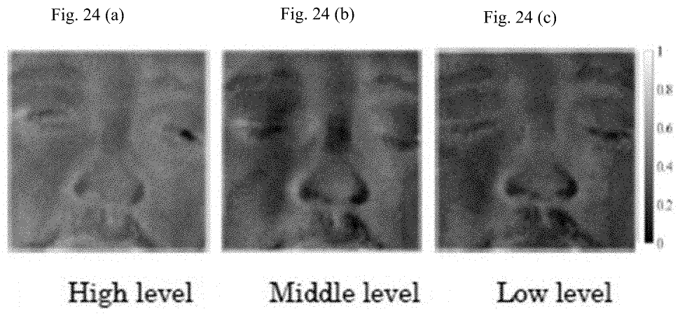

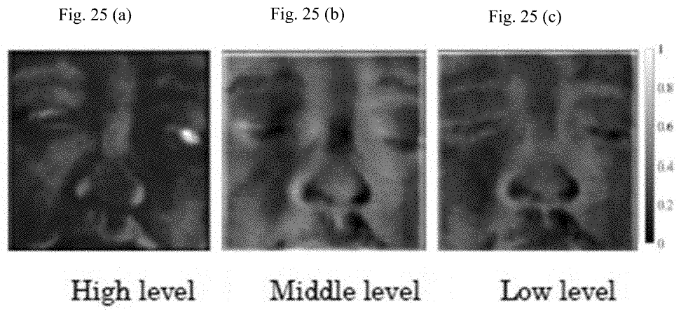

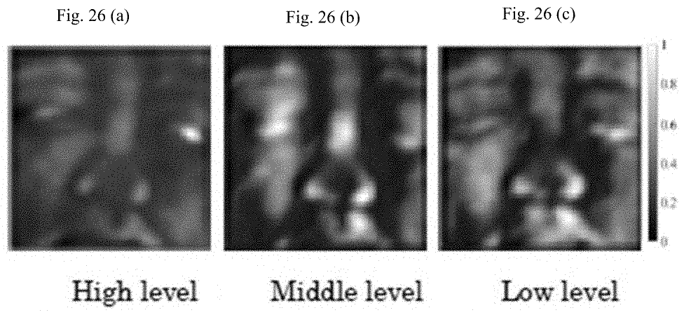

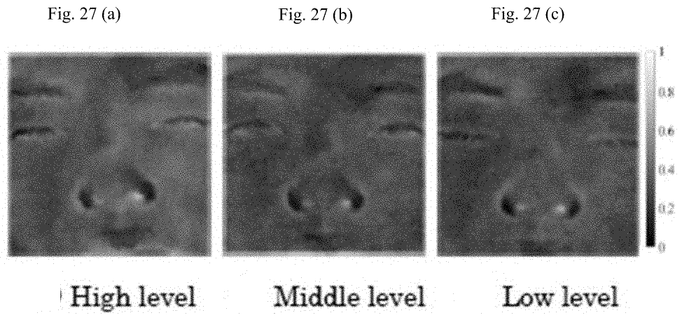

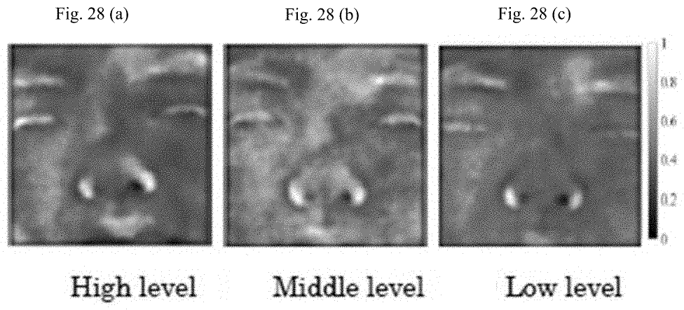

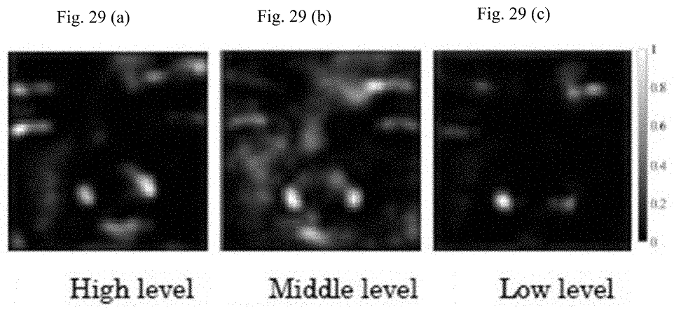

Here, the feature maps of the subject A, who has the highest correct answer rate in the two-stage estimation, and the subject G, who has the lowest correct answer rate in the two-stage estimation are posted. FIGS. 24(A), 24(B) and 24(C) show the feature map of the first convolutional layer of the subject A, FIGS. 25(A), 25(B) and 25(C) show the feature map of the second convolutional layer, and FIGS. 26(A), 26(B) and 26(C) show the feature map of the third convolutional layer with respect to the input of the facial skin temperature distribution at each blood pressure stage. In addition, FIGS. 27(A), 27(B) and 27(C) show the feature map of the first convolutional layer of the subject G, FIGS. 28(A), 28(B) and 28(C) the feature map of the second convolutional layer, and FIGS. 29(A), 29(B) and 29(C) show the feature map of the convolutional layer 3 with respect to the input of the facial skin temperature distribution at each blood pressure stage. However, FIGS. 24(A), 24(B) and 24(C) to FIGS. 29(A), 29(B) and 29(C) show the feature maps with the largest activation among the feature maps in the filters of the convolution layers, the maximum value of the color bar of the feature map indicates the maximum value of the activation of all the feature maps of the same layer, and the minimum value of the color bar indicates the minimum value of activation of all the feature maps of the same layer.

When Subject A had a blood pressure stage which was the Middle level, activation was observed around both the eyes, the nasal dorsum, the nasal wings, and the upper lip (see FIG. 26B). On the other hand, when the blood pressure level was the Low level, activation was observed around the left eye, the nasal wing, and the upper lip (see FIG. 26C). When the blood pressure stage was the High level, strong activation was not observed in the places that were strongly activated at the Middle level and the Low level.

In the subject G, activation was observed in all the blood pressure stages around the eyebrows, the eyes, and the nasal wings (see FIG. 29). When the blood pressure stage was the High level and the Middle level, activation was observed around not only the eyebrows, the eyes, and the nasal wings but also on the lips. When the blood pressure stage was the Middle level, activation was also observed on the right cheek (See FIGS. 29 A and 29B).

Table 8 shows the face regions that were activated in the feature maps of the third convolutional layers of all the subjects with response to the input of the facial skin temperature distribution at each blood pressure stage. It was shown that feature regions appearing on the face changed depending on the blood pressure stage in each subject. Furthermore, it was shown that feature regions appearing on the faces among the subjects were different from each other. It is conceivable that a factor causing the difference between the feature regions appearing on the face among the subjects is a difference between the shapes of the faces and the blood vessel structures in the face. In deep learning, it was suggested that blood pressure stage estimation could be realized by capturing the features of appearing regions changing depending on the blood pressure stages. Furthermore, it was suggested that individuality must be considered to construct a blood pressure stage estimation model because the feature regions appearing on the faces are different from each other among the subjects.

TABLE 8

| Sub. | High level | Middle level | Low level |
|---|---|---|---|
| A | Left eye | Both eyes, nasal | Left eye, nasal wings, |
| | | dorsum, nasal wings, | upper lip |
| | | upper lip | |
| B | Corners of eyes, | Corners of eyes, nasal | Apex of nose, nasal |
| | nasal wings, lips | wings, right cheek, | dorsum, nasal wings, |
| | | lips | lips |
| C | lips | Corner of right eye, | Corner of left eye, left |
| | | lips | cheek |
| D | Right eyebrow | Apex of nose, lips | Apex of nose, right |
| | | | cheek, lips |
| E | Nasal dorsum, | Left nasal wing, left | Both eyebrows, whole |
| | right nasal wing | cheek | nose, both cheeks, lips |
| F | Left eyebrow, | Apex of nose, nasal | Left eyebrow, nasal |
| | nasal wings, lips | wings, lips | dorsum, nasal wings, |
| | | | left cheek, lips |
| G | Both eyebrows, | Both eyebrows, both | Both eyebrows, eyes, |
| | both eyes, nasal | eyes, nasal wings, | nasal wings |
| | wings, lips | right cheek, lips | |

A skin temperature depends on a skin blood flow that changes due to sympathetic nervous system activation that controls contraction of an anterior capillary sphincter, but there is a hysteresis characteristic because relaxation of the anterior capillary sphincter requires time longer than the of the contraction. It is conceivable that the timing of skin temperature change may be delayed in comparison with a blood pressure change due to the hysteresis characteristic. In order to realize more accurate blood pressure sensing, it is necessary to consider the time difference between the blood pressure change and the skin temperature change. On the other hand, since pulse wave information changes instantaneously when a blood pressure changes, pulse wave propagation time and the like are used as an index of the blood pressure. In recent years, researches to acquire a facial pulse wave component from a face visible image in a non-contact manner has been also made. It is conceived that simultaneous use of the skin temperature distribution and the facial pulse wave component, that is, simultaneous use of the temperature information and the pulse wave information will lead to the realization of more accurate non-contact blood pressure sensing.

### 5. Conclusion

In this experiment, to realize continuous monitoring of vital signs using a non-contact sensing technique, deep learning algorithms were applied to a facial skin temperature distribution measured in a state in which the blood pressure was changed to extract features related to the blood pressure, and an attempt to construct an individual model estimating a blood pressure stage on the basis of the features was made. As a result, the correct answer rate was 80% or more in the 2-stage estimation and about 65 to 85% in the 3-stage estimation. Furthermore, it was shown that the feature regions appearing on the face changed depending on the blood pressure stages in the subjects, and the feature regions appearing on the faces were different from each other among the subjects.

### INDUSTRIAL APPLICABILITY

The blood pressure estimation system according to the present invention is capable of instantaneously estimating the blood pressure of a subject in a non-contact manner. Therefore, the blood pressure estimation system is applicable to wide-range technical fields such as a means for estimating the blood pressure of the subject in his/her daily life and a means for estimating the blood pressure of a driver who is driving an automobile.

### REFERENCE NUMERALS

100 blood pressure estimation system
110 face image acquisition device (face image acquisition unit)
120 blood pressure estimation device (blood pressure estimation unit)
121 correlation data storage unit
122 spatial feature amount extraction unit
123 blood pressure determination unit
124 estimated blood pressure value output unit
150 blood pressure estimation system
160 face image acquisition device (face image acquisition unit)
170 blood pressure estimation device (blood pressure estimation unit)
171 correlation data storage unit
172 Spatial feature amount extraction unit
172A weighted time series calculation unit
172B weighted time series differential value calculation unit
173 blood pressure determination unit
174 estimated blood pressure value output unit
200 blood pressure estimation system
210 face Image acquisition device (face image acquisition unit)
220 blood pressure estimation device (blood pressure estimation unit)
221 determination feature amount storage unit
222 spatial feature amount extraction unit
223 blood pressure stage determination unit
224 estimated blood pressure stage output unit
230 learning device (machine learning unit)
231 learning data storage unit
232 feature amount extraction unit
233 feature amount learning unit
CD correlation data
EV estimated value
ES estimation result
FI face image
FTI face thermal image
FVI face visible image
P subject
TM learned model

1. A blood pressure estimation system comprising:
a face image acquisition unit that acquires a face image of a subject in a non-contact manner, and
a blood pressure estimation unit that estimates a blood pressure of the subject based on a spatial feature amount of the face image.

2. The blood pressure estimation system according to claim 1, wherein
the blood pressure estimation unit includes
a correlation data storage unit that stores correlation data indicating a relationship between a weighted time series of an independent component of the face image and a blood pressure,
a spatial feature amount extraction unit that extracts a weighted time series of the independent component of the face image as a spatial feature amount by analyzing the independent component of the face image acquired by the face image acquisition unit,
a blood pressure determination unit that determines the blood pressure value corresponding to the weighted time series extracted by the spatial feature amount extraction unit on the basis of the correlation data, and
an estimated blood pressure value output unit that outputs the value determined by the blood pressure determination unit as an estimated value of the blood pressure of the subject.

3. The blood pressure estimation system according to claim 1, wherein
the blood pressure estimation unit includes
a correlation data storage unit that stores correlation data showing the weighted time series of the independent component of the face image and a relationship between a differential value of the weighted time series and the blood pressure,
a weighted time series calculation unit that calculates a weighted time series of the independent components of the face image as the spatial feature amount by analyzing the independent component of the face image acquired by the face image acquisition unit,
a weighted time series differential value calculation unit that calculates a differential value of the weighted time series calculated by the weighted time series calculation unit,
a blood pressure determination unit that determines a blood pressure value corresponding to the weighted time series calculated by the weighted time series calculation unit and the differential value of the weighted time series calculated by the weighted time series differential value calculation unit from the correlation data, and
an estimated blood pressure value output unit that outputs the value determined by the blood pressure determination unit as an estimated value of the blood pressure of the subject.

4. The blood pressure estimation system according to claim 3, wherein
the differential value of the weighted time series includes a first-order differential value and a second-order differential value of the weighted time series, and
the weighted time series differential value calculation unit calculates the first-order differential value and the second-order differential value of the weighted time series.

5. The blood pressure estimation system according to any one of claim 2, wherein
the face image is a face thermal image or a face visible image.

6. The blood pressure estimation system according to claim 1, wherein
the blood pressure estimation unit includes
a determination feature amount storage unit that stores a determination spatial feature amount corresponding to a blood pressure stage consisting of two stages or three stages,
a spatial feature amount extraction unit that extracts the spatial feature amount of the face image acquired by the face image acquisition unit,
a blood pressure stage determination unit that determines the blood pressure stage of the subject based on the spatial feature amount extracted by the spatial feature amount extraction unit and the determination spatial feature amount, and
an estimated blood pressure stage output unit that outputs the determination result by the blood pressure stage determination unit as an estimation result of the blood pressure stage of the subject.

7. A blood pressure estimation system according to claim 6, wherein
the determination spatial feature amount stored in the determination feature amount storage unit is a spatial feature amount extracted by a machine learning unit, and
the machine learning unit includes
a learning data storage unit that stores a plurality of learning face images labeled corresponding to blood pressure stages consisting of the two stages or the three stages, respectively,
a feature amount extraction unit that extracts the spatial feature amount of the learning face image using a learned model, and
a feature amount learning unit that changes network parameters of the learned model based on a relationship between the extraction result obtained by the feature amount extraction unit and the label attached to the learning face image serving as an extraction target thereof such that the extraction accuracy of the spatial feature amount by the feature amount extraction unit becomes high.

8. The blood pressure estimation system according to claim 7, wherein
the face image is a face thermal image or a face visible image.

9. A learning device comprising:
a learning data storage unit that stores a plurality of learning face images labeled corresponding to blood pressure stages consisting of two stages or three stages;
a feature amount extraction unit that extracts a spatial feature amount of the learning face image using a learned model; and
a feature amount learning unit that changes network parameters of the learned model based on a relationship between an extraction result obtained by the feature amount extraction unit and the label attached to the learning face image serving as an extraction target thereof such that extraction accuracy of the spatial feature amount by the feature amount extraction unit becomes high.

10. A blood pressure estimation method comprising:
providing the blood pressure estimating system of claim 1;
acquiring a face image of a subject; and
estimating a blood pressure of the subject based on a spatial feature amount of the face image.

11. The blood pressure estimation method according to claim 10, wherein
the blood pressure estimation step includes
storing correlation data showing a relationship between a weighted time series of an independent components of the face image and a blood pressure,
extracting the weighted time series of the independent components of the face image as the spatial feature amount by analyzing the independent components of the face image of the subject;
determining the blood pressure value corresponding to the weighted time series extracted in the spatial feature amount extraction step on the basis of the correlation data, and
outputting a determination result obtained by the blood pressure determination step as an estimated value of the blood pressure of the subject.

12. The blood pressure estimation method according to claim 10, wherein
the blood pressure estimation step includes
storing correlation data showing the weight time series of the independent components of the face image and a relationship between a differential value thereof and a blood pressure;
calculating a weighted time series of the independent components of the face image as the spatial feature amount by analyzing the independent components of the face image acquired in the face image acquisition step,
calculating a differential value of the weighted time series calculated by the weighted time series calculation step,
determining, from the correlation data, a blood pressure value corresponding to the weighted time series calculated by the weighted time series calculation step and the differential value of the weighted time series calculated by the weighted time series differential value calculation unit; and
outputting the value determined by the blood pressure determination step as an estimated value of the blood pressure of the subject.

13. The blood pressure estimation method according to claim 12, wherein
the differential value of the weighted time series includes a first-order differential value and a second-order differential value of the weighted time series, and
the weighted time series differential value calculation step is a step of calculating the first-order differential value and the second-order differential value of the weighted time series.

14. The blood pressure estimation method according to claim 10, wherein
the blood pressure estimation step includes
storing a determination spatial feature amount corresponding to blood pressure stages consisting of two stages or three stages,
determining a blood pressure stage of the subject based on the spatial feature amount of the face image of the subject and the spatial feature amount for the determination, and
outputting a determination result obtained by the blood pressure stage determination step as an estimation result of the blood pressure stage of the subject.

15. A learning method comprising:
providing the learning device of claim 9;
storing a plurality of learning face images labeled corresponding to blood pressure stages consisting of two stages or three stages;
extracting a spatial feature amount of the learning face image using a learned model; and
changing network parameters of the learned model such that extraction accuracy of the spatial feature amount by the feature amount extraction step based on a relationship between the extraction result obtained by the feature amount extraction step and the label attached to the learning face image serving as an extraction target thereof.

16. A non-volatile recording medium recording a program for causing the blood pressure estimation system of claim 1 to function as a means for estimating a blood pressure of a subject, comprising:
storing correlation data showing a relationship between a weighted time series of independent components of a face image and a blood pressure;
acquiring the face image of the subject;
extracting the weighted time series of the independent components of the face image as a spatial feature amount of the face image by analyzing the independent components of the face image acquired in the face image acquisition step; and
calculating a blood pressure value corresponding to the weighted time series extracted by the spatial feature amount extraction step from the correlation data to output the value as an estimated value of the blood pressure of the subject.

17. A non-volatile recording medium recording a program for causing a computer to function as a means for estimating a blood pressure of a subject, comprising:
storing correlation data showing the relationship between blood pressure and the weighted time series of the independent component of the face image and its differential value;
calculating a weighted time series of the independent components of the face image as the spatial feature amount by analyzing the independent components of the face image acquired in the face image acquisition step;
calculating a differential value of the weighted time series calculated by the weighted time series calculation step;
determining, from the correlation data, a blood pressure value corresponding to the weighted time series calculated by the weighted time series calculation step and the differential value of the weighted time series calculated by the weighted time series differential value calculation unit; and
outputting the value determined by the blood pressure determination step as an estimated value of the blood pressure of the subject.

18. The program according to claim 17, wherein
the differential value of the weighted time series includes a first-order differential value and a second-order differential value of the weighted time series, and
the weighted time series differential value calculation step is a step of calculating the first-order differential value and the second-order differential value of the weighted time series.

19. A non-volatile recording medium recording a program for causing the blood pressure estimation system of claim 1 to function as a means for estimating a blood pressure of a subject, comprising:
storing a determination spatial feature amount corresponding to a blood pressure stage consisting of two stages or three stages;
acquiring a face image of the subject,
determining a blood pressure stage of the subject based on the face image acquired in the face image acquisition step and the determination spatial feature amount; and
outputting a determination result obtained by the blood pressure stage determination step as an estimation result of the blood pressure stage of the subject.

20. The program according to claim 19, comprising:
storing a plurality of learning face images labeled corresponding to blood pressure stages consisting of two steps or three steps;
extracting a spatial feature amount of the face image from the learning face image using a learned model; and
changing network parameters of the learned model based on a relationship between an extraction result obtained by the feature amount extraction step and a label attached to the learning face image serving as an extraction target thereof such that extraction accuracy of the spatial feature amount obtained by the feature amount extraction step becomes high, wherein
storing the spatial feature amount extracted by the feature amount extraction step.

21. A non-volatile recording medium recording a program for causing the leaning device of claim 9 to function as a learning device for estimating a blood pressure of a subject, comprising:
storing a plurality of learning face images labeled corresponding to blood pressure stages consisting of two steps or three steps;
extracting a spatial feature amount of the learning face image using a learned model; and
changing network parameters of the learned model based on a relationship between an extraction result obtained by the feature amount extraction step and a label attached to the learning face image serving as an extraction target thereof such that extraction accuracy of the spatial feature amount obtained by the feature amount extraction step becomes high.
